(19) United States
(12) Patent Application Publication
(10) Pub. No.: US 2023/0106083 A1
(43) Pub. Date: Apr. 6, 2023

(54) SILICON-FLUORIDE HETEROAROMATIC SYSTEMS FOR APPLICATIONS IN POSITRON EMISSION TOMOGRAPHY (PET) MOLECULAR IMAGING

(71) Applicants: The Regents of the University of California, Oakland, CA (US); FUZIONAIRE DIAGNOSTICS, INC., The Woodlands, TX (US)

(72) Inventors: Jennifer M. Murphy, Los Angeles, CA (US); Maruthi Kumar Narayanam, Los Angeles, CA (US); Anton Toutov, Pasadena, CA (US)

(73) Assignees: The Regents of the University of California, Oakland, CA (US); FUZIONAIRE DIAGNOSTICS, INC., The Woodlands, TX (US)

(21) Appl. No.: 17/604,969

(22) PCT Filed: Apr. 27, 2020
(86) PCT No.: PCT/US20/30074

§ 371 Date: Oct. 19, 2021

Related U.S. Application Data
(60) Provisional application No. 62/958,831, filed on Jan. 9, 2020.
(60) Provisional application No. 62/958,836, filed on Jan. 9, 2020.
(60) Provisional application No. 62/839,396, filed on Apr. 26, 2019.
(60) Provisional application No. 62/839,453, filed on Apr. 26, 2019.

(51) Int. Cl. C07F 7/12

(52) U.S. Cl. CPC C07F 7/122

(57) ABSTRACT

The present invention includes novel compounds and compositions including heteroaromatic Silicon-Fluoride-Acceptors, which are useful for PET imaging, as well as methods for making and using these compounds. The present invention further includes methods of ¹⁸F imaging for PET scanning. In one embodiment the invention is practiced in the form of a kit.

### CROSS-REFERENCE TO RELATED APPLICATIONS

This application claims the benefit under 35 U.S.C. Section 119(e) of co-pending U.S. Provisional Patent Application Ser. Nos. 62/839,396 and 62/839,453, filed on Apr. 26, 2019, and co-pending U.S. Provisional Patent Application Ser. Nos. 62/958,831 and 62/958,836, filed on Jan. 9, 2020, all entitled “SILICON-FLUORIDE HETEROAROMATIC SYSTEMS FOR APPLICATIONS IN POSITRON EMISSION TOMOGRAPHY (PET) MOLECULAR IMAGING”, which applications are incorporated in their entirety by reference herein. This application is related to U.S. Patent Application Publication No. 2018/0346491, filed May 24, 2016, the entire contents of which is incorporated herein by reference.

### TECHNICAL FIELD

The invention relates to agents for imaging targets such as molecules, cells and organs, and compositions and methods for making and using such agents.

### BACKGROUND OF THE INVENTION

The most common ¹⁸F-labeling method for biomolecules to date, utilizes ¹⁸F—SFB, a radiolabeled prosthetic group that reacts with the C-amino group of surface-exposed lysine residues (Liu et al., 2011, Mol. Imaging 10:168; Cai et al., 2007, J. Nucl. Med. 48:304; Olafsen et al., 2012, Tumor Biol. 33:669). In addition, site-specific conjugation using 4-¹⁸F-fluorobenzaldehyde (18-FBA) has also been demonstrated (Cheng et al., 2008, J. Nucl. Med. 49:804). While ¹⁸F—SFB has been successfully used to generated ^{I}T-labeled proteins and peptides, labeling with ¹⁸F—SFB is far from ideal; in addition to its unselective conjugation, its 3-step synthesis and subsequent protein conjugation results in very poor decay-corrected radiochemical yields of 1.4-2.5%.

Silicon fluoride acceptors (SiFAs) are under study as new imaging agents useful for positron emission tomography (see, e.g. Wangler et al., 2012, Appl. Sci., 2:277-302). They can be labeled with the radioisotope flourine-F via a fast and mild ¹⁸F-¹⁹F isotopic exchange reaction (IEX; Kostikov et al., 2012, Nature Protocols, 7:1956-1963). However, the application of SiFA-based PET probes has been hampered by their high intrinsic lipophilicity, originating from bulky tert-butyl groups required for in vivo stabilization of the Si—18 bond. The problems associated with currently known SiFA-imaging probes in preclinical investigations are poor in vivo stability and unfavorable pharmacokinetic behavior.

There is a need in the art for novel precursors for ¹⁸F-labeled compounds, novel ¹⁸F-labeled compounds, and methods for making and using these compounds. The present invention addresses this unmet need.

### SUMMARY OF THE INVENTION

The invention disclosed herein provides Silicon fluoride precursors for making ¹⁸F-labeled compounds, ¹⁸F-labeled Silicon fluoride compounds useful in positron emission tomography (PET), and methods for making and using these compounds. As discussed in detail below, embodiments of the invention include compounds designed to have a constellation of molecular moieties that result in material properties that make these compounds particularly suited for use in positron emission tomography imaging (e.g. material properties that confer desirable hydrophilicity and stability profiles). Embodiments of the invention further include methods of making and using these compounds using reagents and steps that are specifically tailored for use with the compounds disclosed herein.

The invention disclosed herein has a number of embodiments. Embodiments of the invention include compositions of matter useful for making PET probes as well as PET probe compositions. Typically these compositions comprise a compound having an aromatic heterocyclic core comprising a mono- or polycyclic-aromatic chemical moiety featuring one or more heteroatoms. In these compounds, at least one functionality is attached to the aromatic heterocyclic core, wherein the functionality comprises a chemical moiety Q comprising Si; wherein Q is attached to the aromatic heterocyclic core via a covalent chemical bond; an integer number m of atoms or functional groups X associated with Q, wherein m≥0, and each X, if any, is independently chosen such that it can be displaced by a nucleophile, including by [¹⁸F]F⁻; and an integer number n of atoms or functional groups Z covalently bound to Q, wherein n≥0, and each Z is chemically inert and independently chosen such that it stabilizes Q or otherwise protects the functionality from decomposition; and the sum of m, n, and 1 is a value that corresponds to a coordination number specific to Q, wherein the coordination number for Si can be one of: 4, 5, or 6. These compounds further comprise a handle moiety operatively coupled to the aromatic heterocyclic core (e.g. covalently coupled to an atom at position seven on the aromatic heterocyclic core). The handle moiety is adapted to couple the compound to a ligand and typically comprises a functional group selected from the group consisting of: an activated ester, a N-hydroxysuccinimide ester, a maleimide, an aldehyde, a thiol, a nitrile, a disulfide, an alcohol, an isocyanate, a isothiocyanate, an aryl halide, a benzoyl halide, an amine, an azide, an alkyne, a tetrazine, a strained alkyne or a carboxylic acid. In addition, these compounds include a linker operatively coupled to the aromatic heterocyclic core. The linker is adapted to link the aromatic heterocyclic core to the ligand, and typically comprises a functional group selected from an unsubstituted alkyl; an unsubstituted polyethylene glycol, a charged or neutral polyamine; a mixed amino-oxo chain; a polyaromatic polyheteroaromatic group, a charged or neutral polyheteroaromatic group; a bi- or poly-substituted triazole; an imidazole containing group; a peptide, an or amino acid containing moiety or combinations thereof. In certain embodiments of the invention, the compound is coupled to a ligand comprising a peptide, a protein, an enzyme or a small molecule having a molecular weight less than 900 Daltons. Optionally, the compositions further include a pharmaceutically acceptable carrier.

Another embodiment of the invention includes methods of making a heteroaromatic silicon-fluoride compound comprising a [¹⁸F] atom. These methods typically comprise disposing a [¹⁸F]fluoride donor compound within a cartridge comprising a quaternary methyl ammonium so that a [¹⁸F] tetraethyl ammonium fluoride compound is formed; and then eluting the [¹⁸F] tetraethyl ammonium fluoride compound from the cartridge with a solution comprising Tetraethylammonium bicarbonate at a concentration less than 50 μmol. In these methods, the eluted [¹⁸F]tetraethyl ammonium fluoride compound is then dried and combined with a heteroaromatic silicon-fluoride compound precursor (e.g. a compound disclosed herein and comprising an [¹⁹F] atom) with the dry [¹⁸F] tetraethyl ammonium fluoride compound in an organic solvent (e.g. acetonitrile) so that the heteroaromatic silicon-fluoride acceptor compound and the dry [¹⁸F] tetraethyl ammonium fluoride compound exchange F isotopes; and then quenching the isotope-exchange reaction with water, so that the heteroaromatic silicon-fluoride compound comprising the ¹⁸F atom is made. In certain embodiments of these methods, the [¹⁸F] tetraethyl ammonium fluoride compound is eluted from the cartridge with a solution comprising Tetraethylammonium bicarbonate at concentrations less than 50 μmol, for example between 5 μmol and 15 μmol. Typically these methods (are performed at room temperature) and/or obtain a radiochemical conversion of at least 80%. As shown in Table 1 below, embodiments of this methodology have a number of desirable features including the ability to use relatively low loading amounts of heteroaromatic silicon-fluoride compound precursor compounds.

Other embodiments of the invention include methods for imaging a biological target by positron emission tomography. Typically these methods comprise introducing into the target an imaging agent comprising: a composition disclosed herein, wherein F is ¹⁸F; and then imaging the target by a positron emission tomography process such that the biological target is imaged by positron emission tomography. In illustrative embodiments of the invention, the biological target comprises a human organ, human tissue or human cancer cells.

Yet another embodiment of the invention is a kit for ¹⁸F-labeling of a compound disclosed herein, the kit comprising a compound disclosed herein wherein F is ¹⁹F. The kit further includes an ¹⁸F isotopic exchange reagent, and an instruction manual for the use thereof. In illustrative embodiments of the invention, the kit includes a container comprising a nonpolar solution comprising Tetraethylammonium bicarbonate at a concentration between 5 μmol and 20 μmol.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides a schematic of Boron-, phosphine-, and silicon-based building blocks for ¹⁸F-labeling via isotope exchange.

FIG. 2 shows Scheme 1 as discussed in Example 1 below, which is a schematic depicting a synthetic pathway to afford heteroarylsilanes in good yield from commercial heteroarenes, which subsequently underwent fluorination with potassium fluoride and 18-crown-6 in the presence of acetic acid to afford Heteroaromatic silicon fluoride acceptor precursors 1.

FIG. 3 shows Scheme 2 as discussed in Example 1 below, which is a schematic depicting the synthesis of Glycine-functionalized Heteroaromatic silicon fluoride acceptor 6, which can be synthesized in four steps starting from commercially available benzothiophene 2.

FIG. 4 shows Scheme 3 as discussed in Example 1 below, which is a schematic depicting reaction conditions applied to a variety of diverse Heteroaromatic silicon fluoride acceptor radiosynthons, which were prepared via potassium tert-butoxide-catalyzed C—H silylation.

FIG. 5 shows Scheme 4 as discussed in Example 1 below, which is a schematic depicting the synthesis of peptide-based Heteroaromatic silicon fluoride acceptor tracers using a commercial peptide derived from the hormone cholecystokinin, cholecystokinin tetrapeptide (CCK-4). CCK-4 is a small peptide fragment with the sequence H-Trp-Met-Asp-Phe-NH2.

FIGS. 6A and 6B provide data from MicroPET/CT imaging studies. FIG. 6A shows the MicroPET/CT imaging results of experiments where [18F]-17 was injected i.v. into C57BL/6 mice (n=3), and PET images were acquired. FIG. 6B provides graphed data showing representative PET/CT maximum intensity projection images in one mouse and region-of-interest (ROI) analysis of PET data at 1 and 2 h post injection of the radiotracer. Error bars are standard deviations.

FIG. 7 shows a schematic of prosthetic labeling groups in an illustrative benzothiophene heterocyclic SiFA.

FIG. 8 shows a schematic of illustrative working embodiments of the disclosed herein, ones where a benzothiophene structure was used in positron emission tomography (PET) probe development and subsequent imaging studies. The benzothiophene was attached to a peptide via a PEG linker and a polar group to improve biodistribution. The precursor compound was radiolabeled with fluorine-18 via isotopic exchange, purified with C18 cartridge and formulated for in vivo imaging.

FIG. 9 shows a schematic of another working embodiment of the disclosed herein to further illustrate how the moieties/elements of the disclosed molecules can have different three-dimensional architectures. For example, in one embodiment, the architecture/layout can be akin to that shown in this figure one where the biomolecule is in between the Heteroaromatic silicon fluoride acceptor core (with linker) and a polar auxiliary.

FIG. 10 shows data from an analytical HPLC chromatograph of purified benzothiophene-SiFA-peptide conjugate 17. HPLC mobile phase: 10% acetonitrile in water (both with 0.1% TFA) to 90% over 18 min then 95% acetonitrile up to 25 min with flow rate 1.2 mL/min at UV 254 nm.

FIG. 11 shows data from an isotopic exchange and characterization of methyl ((2-(di-tertbutylfluorosilyl) benzo[b]thiophen-7-yl)methyl)glycinate ([18F]-6). Radio-TLC scan (left). Radio-HPLC (right) with 254 nm UV trace of 19F reference standard (upper chromatogram) and radioactivity trace of reaction mixture (lower chromatogram). HPLC mobile phase: 30% Acetonitrile in water (both with 0.1% TFA) to 90% acetonitrile in water over 10 min; then to 95% acetonitrile in water from 13 min to 18 min.

FIG. 12 shows data from an isotopic exchange and characterization of 2-(di-tert-butylfluorosilyl)-1-methyl-1H-indole ([18F]-7). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 8 min; then to 95% acetonitrile in water from 8 min to 25 min.

FIG. 13 shows data from an isotopic exchange and characterization of benzo[b]thiophen-2-yldi-tertbutylfluorosilane ([18F]-8). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 8 min; then to 95% acetonitrile in water from 8 min to 25 min.

FIG. 14 shows data from an isotopic exchange and characterization of 2-(di-tert-butylfluorosilyl)-1-methyl-1H-pyrrolo[2,3-b]pyridine ([18F]-9). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 10 min; then to 95% acetonitrile in water from 10 min to 25 min.

FIG. 15 shows data from an isotopic exchange and characterization of di-tert-butylfluoro(5-pentylfuran-2- yl)silane ([18F]-10). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 10 min; then to 95% acetonitrile in water from 10 min to 25 min.

FIG. 16 shows data from an isotopic exchange and characterization of 1-benzyl-2-(di-tert-butylfluorosilyl)-1H-pyrrole ([18F]-11). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 8 min; then to 95% acetonitrile in water from 8 min to 25 min.

FIG. 17 shows data from an isotopic exchange and characterization of 2-(di-tertbutylfluorosilyl) benzo[b]thiophene-7-carboxylic acid ([18F]-12). Radio-TLC scan (left). Radio-HPLC (right) with 254 nm UV trace of 19F reference standard (upper chromatogram) and radioactivity trace of reaction mixture (lower chromatogram). HPLC mobile phase: 10% Acetonitrile in water to 95% acetonitrile in water over 12 min; then to 95% acetonitrile in water from 12 min to 25 min.

FIG. 18 shows data from a radio-HPLC with 254 nm UV trace (top) and radioactivity trace (lower) of crude reaction mixture after 2 min. HPLC mobile phase: 10% acetonitrile in water (both with 0.1% TFA) to 95% over 15 min then 95% acetonitrile up to 25 min with flow rate 1.2 mL/min.

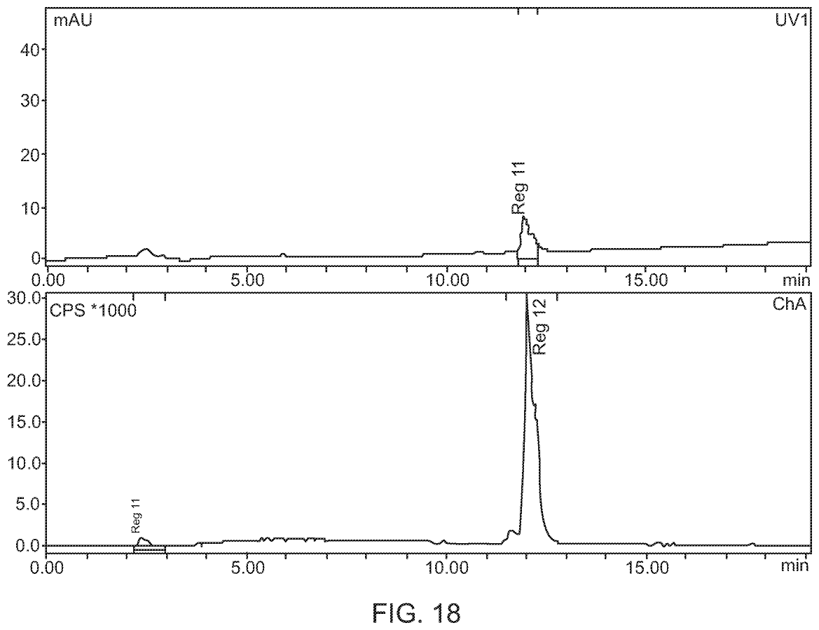

FIG. 19 shows data from a radio-HPLC of formulated peptide [18F]-17 with 254 nm UV trace (top) and radioactivity trace (lower). HPLC mobile phase: 10% acetonitrile in water (both with 0.1% TFA) to 90% over 18 min then to 95% acetonitrile at 25 min with flow rate 1.2 mL/min.

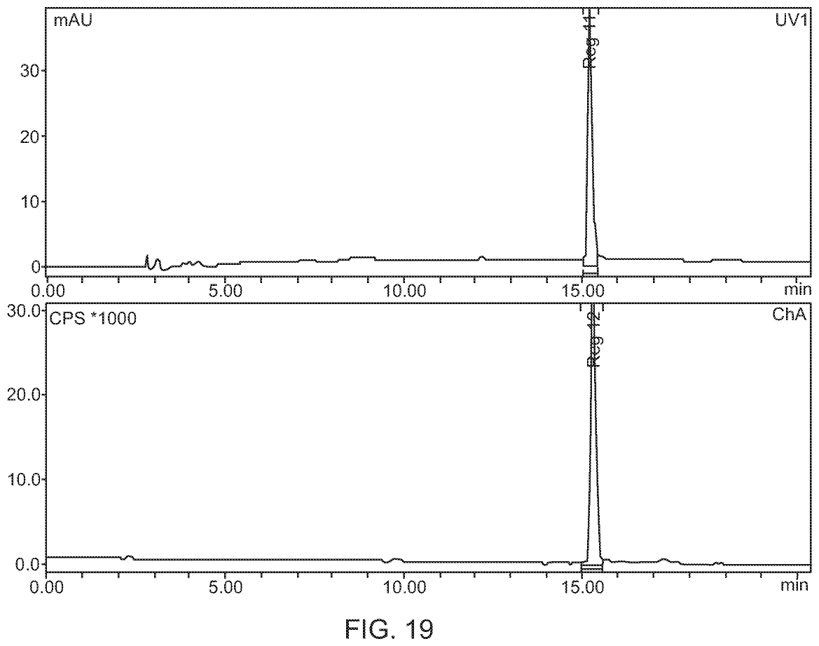

FIG. 20 shows data from a radio-HPLC with 254 nm UV trace of reference standard 17 (top) and radioactive trace of [18F]-17 (lower). HPLC mobile phase: 10% acetonitrile in water (both with 0.1% TFA) to 90% over 18 min then to 95% acetonitrile at 25 min with flow rate 1.2 mL/min.

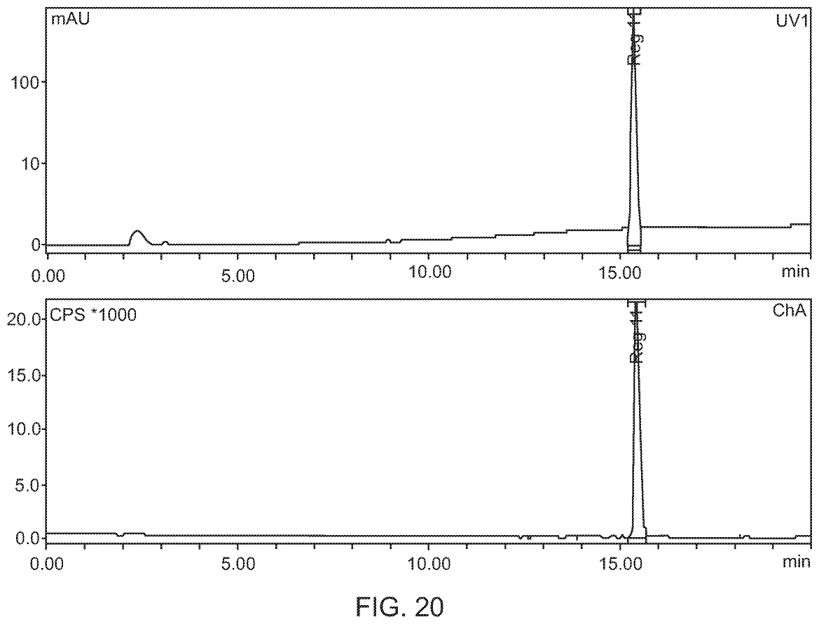

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following provides illustrative embodiments of the invention. All publications mentioned herein are incorporated herein by reference to disclose and describe aspects, methods and/or materials in connection with the cited publications.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of .+−0.20% or .+−0.10%, more preferably .+−0.5%, even more preferably .+−0.1%, and still more preferably .+−0.0.1% from the specified value, as such variations are appropriate to perform the disclosed methods.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. For compositions suitable for administration to humans, the term “pharmaceutically acceptable” is meant to include, but is not limited to, those ingredients described in Remington: The Science and Practice of Pharmacy, Lippincott Williams & Wilkins, 21st ed. (2006).

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the terms “imaging agent,” “imaging probe,” or “imaging compound,” means, unless otherwise stated, a molecule which can be detected by its emitted signal, such as positron emission, autofluorescence emission, or optical properties. The method of detection of the compounds may include, but are not necessarily limited to, nuclear scintigraphy, positron emission tomography (PET), single photon emission computed tomography (SPECT), magnetic resonance imaging, magnetic resonance spectroscopy, computed tomography, or a combination thereof depending on the intended use and the imaging methodology available to the medical or research personnel.

As used herein, the term “biomolecule” refers to any molecule produced by a living organism and may be selected from the group consisting of proteins, peptides, polysaccharides, carbohydrates, lipids, as well as analogs and fragments thereof. Preferred examples of biomolecules are proteins and peptides.

As used herein, the terms “bioconjugation” and “conjugation,” unless otherwise stated, refers to the chemical derivatization of a macromolecule with another molecular entity. The molecular entity can be any molecule and can include a small molecule or another macromolecule. Examples of molecular entities include, but are not limited to, compounds of the invention, other macromolecules, polymers or resins, such as polyethylene glycol (PEG) or polystyrene, non-immunogenic high molecular weight compounds, fluorescent, chemiluminescent radioisotope and bioluminescent marker compounds, antibodies, biotin, diagnostic detector molecules, such as a maleimide derivatized fluorescein, coumarin, a metal chelator or any other modifying group. The terms bioconjugation and conjugation are used interchangeably throughout the Specification.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆) alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and 13 NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. In one embodiment, the alkoxy group is (C₁-C₃) alkoxy, such as ethoxy and methoxy.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

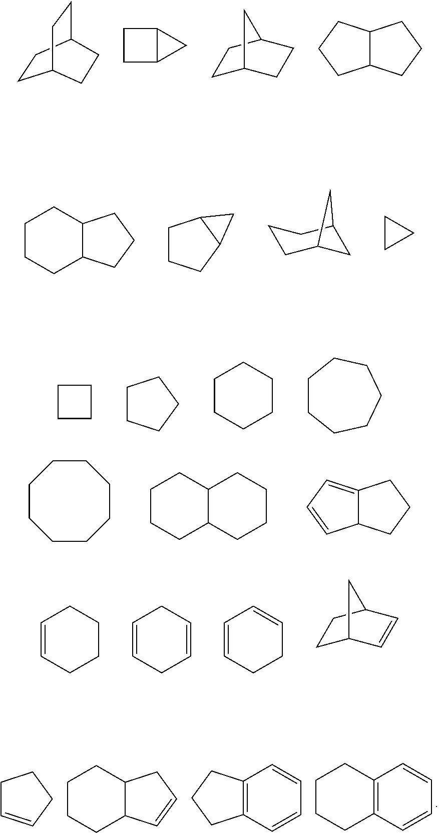

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring.

In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

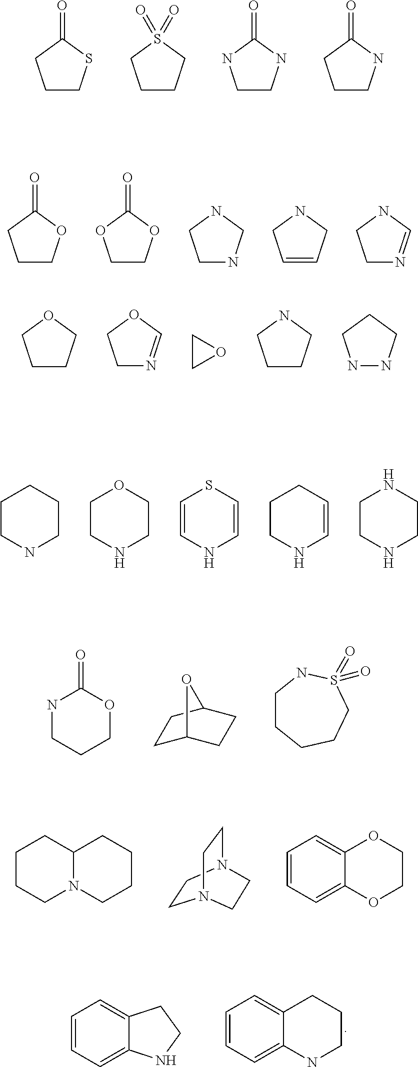

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. In one embodiment, the heteroaryl-(C₁-C₃)alkyl is heteroaryl-(CH₂)—. The term “substituted heteroaryl-(C₁-C₃)alkyl” means a heteroaryl-(C₁-C₃)alkyl functional group in which the heteroaryl group is substituted. In one embodiment, the substituted heteroaryl-(C₁-C₃)alkyl is substituted heteroaryl-(CH₂)—. As used herein, the term “heteroaryl” or “heteroaromatic” refers to a heterocycle having aromatic character. A polycyclic heteroaryl may include one or more rings that are partially saturated. Examples include the following moieties:

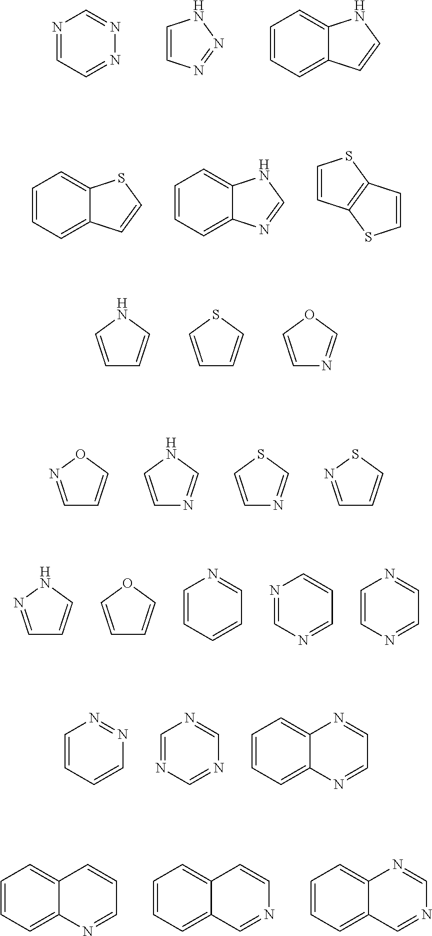

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

As discussed below, embodiments of the present invention provide novel heteroaromatic SiFAs useful for the ¹⁸F-radiolabeling and imaging of biomolecules and methods for making using them. This novel class of heteroaromatic SiFAs significantly improves many aspects of currently available phenyl SiFAs in terms of their preparation and pharmacokinetic properties. As demonstrated herein, the synthesis of heteroaromatic SiFAs does not require the use of highly pyrophoric lithium or magnesium reagents, does not require prefunctionalization of the aryl, can potentially be scaled up to amounts that are of industrial interest, and uses cheaper and more environmentally friendly substrates which aligns with the current goals of sustainable chemistry. The huge variety of available heteroaromatic compounds that can be transformed into SiFAs enables the development of SiFAs with different electronic structures, polarities and free sites for derivatization, advantages which currently available phenyl SiFAs do not have.

As discussed in detail below, the invention provides ¹⁹F precursor heteroaromatic SiFAs. In another embodiment, the invention provides ¹⁸F-labeled compounds derived from such precursor SiFAs. In typical embodiments, the precursors for SiFAs are synthetically accessible by a methodology using potassium tert-butoxide as a catalyst for the silylation of C—H bonds in aromatic heterocycles, methodology described by Toutov et al., Nature, 2015, 518:80-84, which is incorporated by reference herein in its entirety. In another embodiment, the invention provides methods for ¹⁸F-radiolabeling of SiFAs by isotopic exchange. In one such embodiment, the isotopic exchange is performed on various platforms including a commercial radiosynthesizer (ELYXIS, Sofie Biosciences), an in-house developed microfluidic Teflon™-coated chip, and a manual procedure in a sealed glass vial. In another embodiment, the invention provides a kit for ¹⁸F-radiolabeling of SiFAs by isotopic exchange. In another embodiment, the invention provides methods for ¹⁸F-based imaging methods, including, but not limited to, positron emission tomography (PET).

As discussed below, embodiments of the present invention relate to a compound of Formula 1:

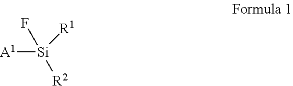

wherein in Formula 1, F is selected from the group consisting of ¹⁹F and ¹⁸F; A¹ is a monocyclic or bicyclic heteroaryl ring optionally substituted with 0-4 R^{a} groups; R^{a} is selected, at each independent occurrence, from the group consisting of null, H, F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{cc}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, or independent a groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R^{c}, R^{d} or R^{e} can optionally be joined to form additional rings; and R¹ and R² are each independently an alkyl group.

In one embodiment, A¹ is selected from the group consisting of indole, 7-azaindole, benzothiophene, furan, pyrrole, pyrazole, imidazole, and pyridine. In another embodiment, R¹ and R² are tert-butyl groups. In another embodiment, A¹ is selected from the group consisting of indole, 7-azaindole, benzothiophene, furan, pyrrole, pyrazole, imidazole, and pyridine, and R¹ and R² are tert-butyl groups. In another embodiment, the compound is selected from the group consisting of:

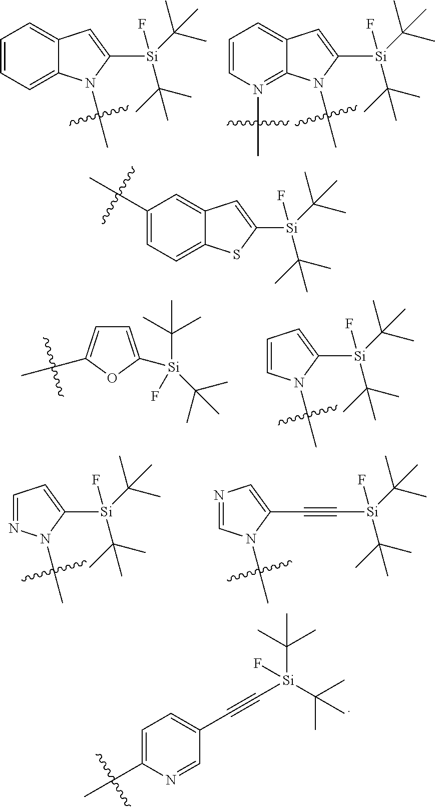

In another embodiment, the compound is selected from the group consisting of:

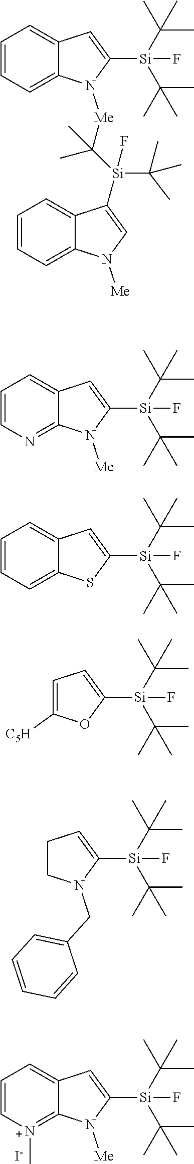

The present invention also relates to a compound of Formula 2:

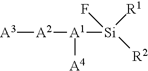

wherein in Formula 2, F is selected from the group consisting of ¹⁹F and ¹⁸F; A¹ is a monocyclic or bicyclic heteroaryl ring optionally substituted with 0-4 R^{a} groups; A² is a linker; A³ is a moiety capable of chemical conjugation or bioconjugation;
A⁴ is a moiety comprising a polar auxiliary moiety or compound that may optionally contain a charge; R^{a} is selected, at each independent occurrence, from the group consisting of null, H, F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, or independent R^{a} groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R^{c}, R^{d} or R^{e} can optionally be joined to form additional rings; and R¹ and R² are each independently an alkyl group.

In one embodiment, A¹ is selected from the group consisting of indole, 7-azaindole, benzothiophene, furan, pyrrole, pyrazole, imidazole, and pyridine. In another embodiment, R¹ and R² are tert-butyl groups. In another embodiment, A¹ is selected from the group consisting of indole, 7-azaindole, benzothiophene, furan, pyrrole, pyrazole, imidazole, and pyridine, and R¹ and R² are tert-butyl groups. In another embodiment, A² includes at least one of an unsubstituted alkyl, an unsubstituted polyethylene glycol (PEG), and a bisubstituted triazole. In another embodiment, A³ is selected from the group consisting of an N-hydroxysuccinimide (NHS) ester and maleimide.

In one embodiment, the compound of Formula 2 is a compound of Formula 3:

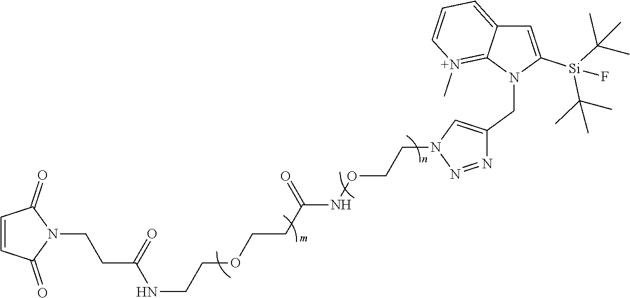

wherein in Formula 3, m and n are each independently an integer selected from the group consisting of 0, 1, 2, 3, 4, 5, and 6. In another embodiment, m=2 and n=3.

The present invention also relates to a compound of Formula 4:

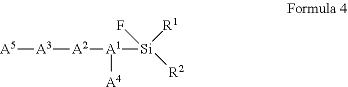

wherein in Formula 4,

F is selected from the group consisting of ¹⁹F and ¹⁸F; A¹ is a monocyclic or bicyclic heteroaryl ring optionally substituted with 0-4 R^{a} groups; A² is a linker; A³ is a moiety capable of chemical conjugation or bioconjugation; A⁴ is a moiety comprising a polar auxiliary moiety that may optionally contain a charge; A⁵ is a moiety comprising a disease targeting molecule or biomolecule; R^{a} is selected, at each independent occurrence, from the group consisting of null, H, F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{e}, C(═O)NR^{c}R^{d}, C(═O)OR, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R, S(═O)₂R^{c}, and S(═O)₂NR^{c}R^{d}, or independent R^{a} groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R^{c}, R^{d} or R^{e} can optionally be joined to form additional rings; and R¹ and R² are each independently an alkyl group.

In one embodiment, A¹ is selected from the group consisting of indole, 7-azaindole, benzothiophene, furan, pyrrole, pyrazole, imidazole, and pyridine. In another embodiment, R¹ and R² are tert-butyl groups. In another embodiment, A¹ is selected from the group consisting of indole, 7-azaindole, benzothiophene, furan, pyrrole, pyrazole, imidazole, and pyridine, and R¹ and R² are tert-butyl groups. In another embodiment, A² includes at least one of an unsubstituted alkyl, an unsubstituted polyethylene glycol (PEG), or a bisubstituted triazole. In another embodiment, A³ is selected from the group consisting of an NHS ester, a maleimide, an amide, and a maleimide-thiol adduct.

### Handle and Linker Moieties of the Invention

Embodiments of the current invention utilize heteroaromatic Silicon-Fluoride Acceptors (SiFAs) synthesized to include selected moieties that confer desirable material properties. In particular, certain embodiments of the invention focus on molecules having a specific constellation of chemical moieties/elements observed to provide the compounds with material properties that make them particularly suited for use in PET technologies (e.g. desirable in vivo hydrophilicity and stability profiles). These moieties include linker moieties that function to couple a SiFA core to a biomolecule such as a polypeptide as well as a “handle” moiety. The term “handle” as used in the context of the moieties/elements of the SiFA compounds disclosed herein refers the moieties on the SiFA compounds to which a biological molecule of interest is attached (i.e. a moiety capable of chemical conjugation or bioconjugation to a biological molecule such as a peptide). In certain embodiments of the invention, the handle can be part of a linker and function to facilitate attachment of the linker to the biomolecule (e.g. where a linker moiety is attached to a core using a handle moiety that further facilitates attachment of the linker to the biomolecule).

The successful applicability of heterocyclic SiFAs critically depends on introduction of appropriate functional groups on to the heterocyclic molecule to produce a functional PET molecule. In this context, specific constellations of elements that result in PET molecules having the desired properties can be hard to predict. We have discovered that certain constellations of elements do not work, while others work unexpectedly well. In the illustrative embodiments of the invention discussed in this section, initial C—H silylation of heterocycles were achieved by conventional methodology using potassium tert-butoxide catalyst (see, e.g. Toutov, A. A. et al. Nature, 2015, 518, 80-84). SiFAs compounds can be synthesized to include moieties that provide them with material properties that make them particularly useful in technologies such as PET. Aromatic heterocycles included in this group of embodiments of the invention include derivatives of indole, 7-azaindole, benzothiophene, furan, pyrrole, pyrazole, imidazole, pyridine and the like. The schematic immediately below identifies a general structure for embodiments of the invention.

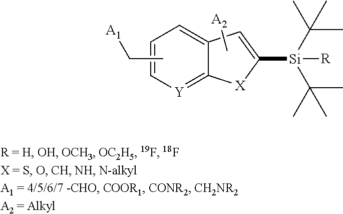

Embodiments of this group of inventions provides heteroaromatic SiFAs that are attached with selected functional handle and/or linker moieties in order to conjugate this core structure to agents such as disease targeting (bio)molecules (e.g. ligands, peptides, proteins, enzymes, antibodies, small molecules and the like). Conjugation to a (bio)molecule can be achieved via a conventional method in this art, such as one using a N-hydroxysuccinimide (NHS) ester, maleimide, or known click chemistry methodology. The conjugation of functionalized heterocyclic SiFAs to desired peptides or proteins along with additional elements such as polar auxiliary and chelator moieties and agents that improve the stability and/or bioavailability of such embodiments, make these embodiments of the invention function as improved PET imaging probes.

Embodiments of the invention include compositions of matter useful for making PET probes as well as PET probe compositions. Typically these compositions comprise a compound having an aromatic heterocyclic core comprising a mono- or polycyclic-aromatic chemical moiety featuring one or more heteroatoms. In these compounds, at least one functionality is attached to the aromatic heterocyclic core, wherein the functionality comprises a chemical moiety Q comprising Si; wherein Q is attached to the aromatic heterocyclic core via a covalent chemical bond; an integer number m of atoms or functional groups X associated with Q, wherein m≥0, and each X, if any, is independently chosen such that it can be displaced by a nucleophile, including by [¹⁸F]F⁻; and an integer number n of atoms or functional groups Z covalently bound to Q, wherein n≥0, and each Z is chemically inert and independently chosen such that it stabilizes Q or otherwise protects the functionality from decomposition; and the sum of m, n, and 1 is a value that corresponds to a coordination number specific to Q, wherein the coordination number for Si can be one of 4, 5, or 6. These compounds further comprise a handle moiety operatively coupled to the aromatic heterocyclic core (e.g. a carboxylic acid moiety or the like on position 7 of the aromatic heterocyclic core), wherein, the handle moiety is adapted to couple the compound to a ligand and the handle moiety comprises a functional group selected from the group consisting of: an activated ester, a N-hydroxysuccinimide ester, a maleimide, an aldehyde, a thiol, a nitrile, a disulfide, an alcohol, an isocyanate, a isothiocyanate, an aryl halide, a benzoyl halide, an amine, an azide, an alkyne, a tetrazine, a strained alkyne or a carboxylic acid. In addition, these compounds include a linker operatively coupled to the aromatic heterocyclic core. As used herein, the term “operatively coupled” refers to embodiments of the invention where a moiety is directly coupled to the aromatic heterocyclic core as well as embodiments of the invention where the moiety is indirectly coupled to the aromatic heterocyclic core, for example via other atoms in the compounds of the invention. In certain embodiments of the invention, a first moiety such as the handle moiety is directly coupled to the aromatic heterocyclic core while a second moiety such as the linker moiety is indirectly coupled to the aromatic heterocyclic core. Typically in such embodiments, wherein the linker is adapted to link the aromatic heterocyclic core to the ligand, and the linker moiety comprises a functional group selected from an unsubstituted alkyl; an unsubstituted polyethylene glycol, a charged or neutral polyamine; a mixed amino-oxo chain; a polyaromatic polyheteroaromatic group, a charged or neutral polyheteroaromatic group; a bi- or poly-substituted triazole; an imidazole containing group; a peptide, an or amino acid containing moiety or combinations thereof.

In certain embodiments of the invention, this compound is of the general formula:

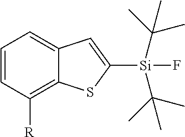

wherein R comprises the handle moiety.

In some embodiments of the invention, the compound is coupled to a ligand comprising a peptide, a protein, an enzyme or a small molecule having a molecular weight less than 900 daltons, for example by site selective chemical conjugation of the ligand to the compound (e.g. so as to function as a PET imaging agent). Optionally, the compositions further include a pharmaceutically acceptable carrier. In certain embodiments of the invention the compositions include an element selected to modulate hydrophilicity, for example so that the compound exhibits a negative charge at physiological pH. For example, certain embodiments of the invention can include a chelator such as a non-metalated hydrophilic metal chelator. In illustrative embodiments of the invention, the chelator comprises DOTA (1,4,7,10-tetraazacyclododecane-1,4,7,10-tetraacetic acid) or NOTA (1,4,7-Triazacyclononane-1,4,7-triacetic acid). Embodiments of the invention can also include a polar auxiliary moiety operatively coupled to the compound (e.g. so as to modulate hydrophilicity of the compound).

The compounds of the invention can have a number of three-dimensional architectures. For example, in some embodiments of the invention, the compound has the general formula:

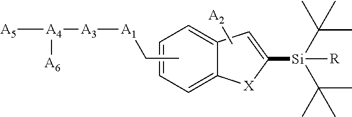

Where: A1 comprises the handle moiety; A2 comprises an unsubstituted alkyl group; A3 comprises an unsubstituted polyethylene glycol or a bisubstituted triazole; A4 comprises the ligand; A5 comprises a chelator; A6 comprises a polar auxiliary moiety; and R comprises a fluorine atom.

The following schematic shows the structure of a selected embodiment of the invention.

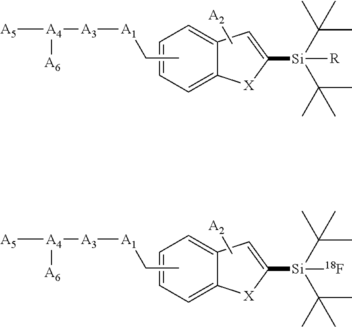

In the schematic immediately above, A1 comprises a functional handle moiety; A2 comprises an unsubstituted alkyl group(s), A3 comprises an unsubstituted polyethylene glycol or a bisubstituted triazole; A4 comprises a disease targeting biomolecule or the like such as cyclic or branched peptides or proteins; A5 comprises a chelator such as DOTA, NOTA and the like conjugated through amide linkage; and A6 comprises a polar auxiliary moiety that may or may not contain a charge.

The selected working embodiments disclosed in this section are useful in ¹⁸F-radiolabeling of complex molecules such as peptides and proteins by isotopic exchange. This labelling methodology can be performed on various platforms including a commercial radiosynthesizer (e.g. ELYXIS, Sofie Biosciences), an in-house developed microfluidic Teflon®-coated chip, and a manual procedure in a sealed glass vial etc.

The functionalized SiFAs designed by the inventors and described herein exhibit improved aspects over currently available phenyl SiFAs in terms of their synthetic preparation and pharmacokinetic properties. For example, some of the most urgent problems that are associated with currently known SiFA-imaging probes in preclinical investigations are poor in vivo stability and unfavorable pharmacokinetic behavior. Currently, the stability issue is conventionally addressed by adding two bulky tert-butyl groups on silicon. The functionalized heterocyclic SiFAs addresses such problems by enabling the facile synthesis of a variety of desired conjugates with variable polarities. For example, we have discovered that addition of a PEGylated linker adds stability and improved lipophilicity to the overall molecule.

However, as disclosed herein, in order to enhance bioavailability as a PET imaging probe, the PET molecule needs to have a polar group (free carboxylates) or charged group (such as quaternary ammonium ion, NR4+) to improve the overall lipophilicity (increase polarity of the molecule). In this context, the huge variety of available heteroaromatic compounds that can be transformed into SiFAs enables the future synthesis and identification of SiFAs with different electronic structures, polarities and free sites for derivatization (at any carbon on the heteroaromatic ring system). Currently available phenyl SiFAs do not have this advantage.

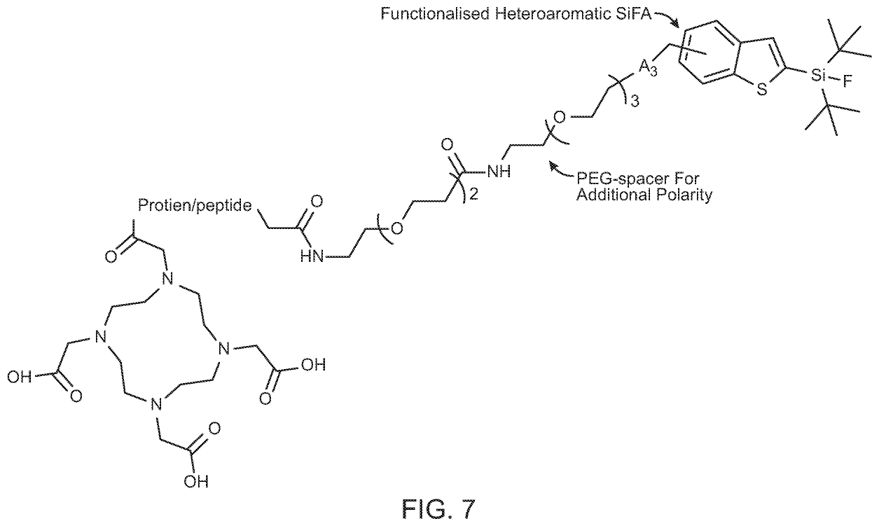

One example of prosthetic labeling groups based on benzothiophene heterocyclic SiFA is shown in FIG. 7. The general structure in FIG. 7 comprises of benzothiazole SiFA conjugated with targeted peptide along with polar auxiliaries and chelators to improve the bioavailability of PET probe. The novel class of heteroaromatic SiFAs described in this section can be labeled with the PET isotope ¹⁸F using various platforms. In all cases, the pure labeling products are obtained by a simple cartridge purification (C18 or alumina).

### Chemistry of the Handle Moiety to Functionalize the Core Heteroaromatic Silicon-Fluoride Acceptor

As illustrated by the schematic immediately below, a handle moiety (R) in embodiments of the invention can be various functional groups attached, for example to the 7-position of the benzothiophene core such as activated ester (N-hydroxysuccinimide ester or other activating group), maleimide, carboxylic acid, alcohol, aldehyde, amine, nitrile, amide, thiol, isothiocyanate, isocyanate, halide, alkyne, azide, tetrazine, strained alkyne. In certain embodiments of the invention, a silyl group is attached at the 2-position of the benzothiophene core and a handle moiety is attached to the 7-position of the benzothiophene core.

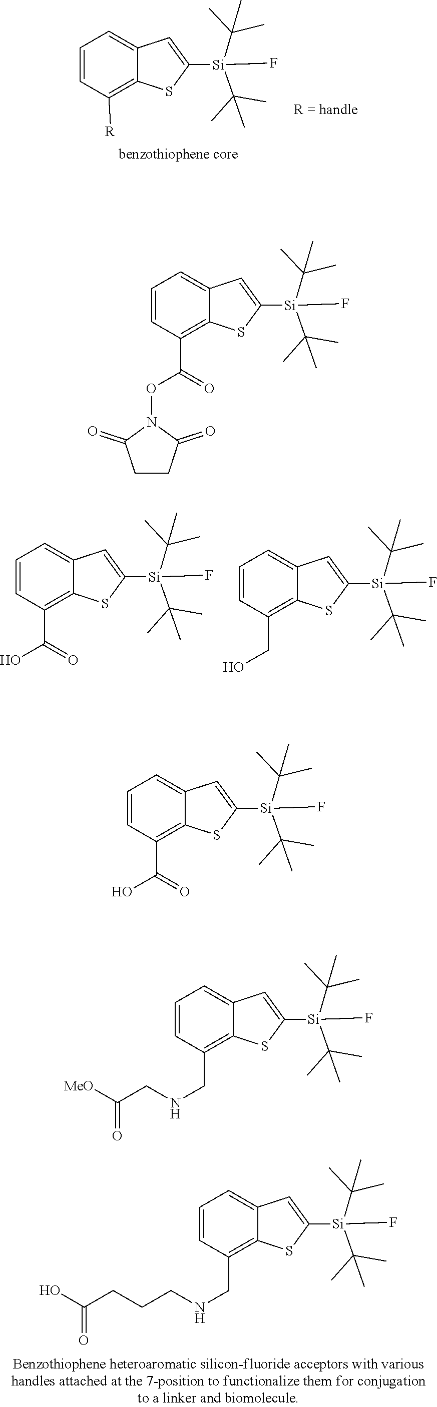

The above-noted schematic provides illustrative non-limiting examples of embodiments of the invention having functional groups attached to the 7-position. In other embodiments of the invention, the embodiments of the invention have different three-dimensional architectures. For example, in other embodiments of the invention, the core potion of the molecule can be functionalized at the 5-position as shown immediately below.

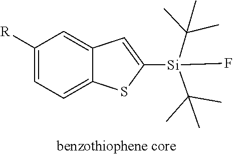

Chemistry of Moiety Linking the Core Heteroaromatic Silicon Fluoride Acceptor with a Biomolecule

In typical embodiments of the invention, a linker moiety/compound is used to attach the core Heteroaromatic silicon fluoride acceptor molecule to a biomolecule used in PET imaging, such as a polypeptide or the like. In certain embodiments of the invention, this linker must comprise a PEG chain of some length to ensure the stability of the overall molecule (without the PEG chain, the molecule is not stable in solution). The linker can also include various charged amino acid groups such as aspartic acid, glutamic acid, histidine; charged polyamine or heteroaromatic compounds; triazole or imidazole compounds or the like.

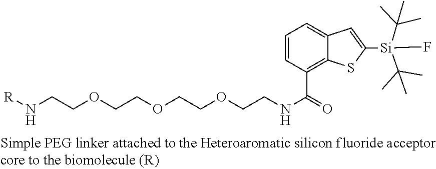

In certain embodiments of the invention, we developed the use of the amino acid lysine as a core linker to link together the three components: polar auxiliary, Heteroaromatic silicon fluoride acceptor biomolecule. These can be linked interchangeably via the lysine core shown below.

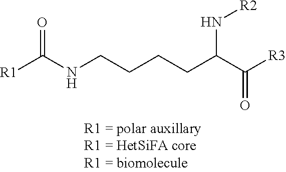

The polar auxiliary moiety is an important component in the embodiments of the invention discussed in this section as it can be used to improve the biodistribution of the molecule (which is to be applied as a PET imaging probe) and, for example, greatly enhance renal clearance which is desirable for a PET probe. Various polar auxiliaries can be used such as charged amino acids, aspartic acid, glutamic acid, histidine; charged polyamine groups; glycosyl analogue; or non-metalated hydrophilic metal chelator such as DOTA (1,4,7,10-tetraazacyclododecane-1,4,7,10-tetraacetic acid) or NOTA (1,4,7-Triazacyclononane-1,4,7-triacetic acid). One polar auxiliary moiety that we unsuccessfully attempted to generate was one that installed a cysteic acid residue. We instead therefore used DOTA as a polar auxiliary moiety.

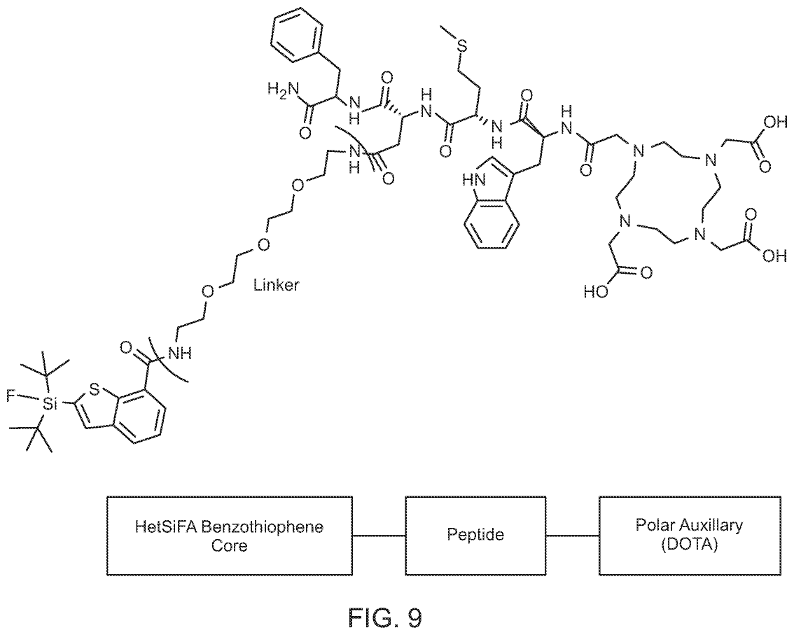

The moieties/elements of the disclosed molecules can have different three-dimensional architectures. For example, in one embodiment the architecture/layout can be akin to that shown in FIG. 9, one where the biomolecule is in between the Heteroaromatic silicon fluoride acceptor core (with linker) and the polar auxiliary. In another embodiment, the architecture/layout can be one where the polar auxiliary, peptide and Heteroaromatic silicon fluoride acceptor core are linked via lysine (described above). In another embodiment, the polar auxiliary moiety can be directly coupled to the Heteroaromatic silicon fluoride acceptor core (e.g. before or after the linker) with the peptide attached at the end.

### Methods of Making a Heteroaromatic Silicon-Fluoride Compound Comprising a [¹⁸F] Atom

In one embodiment, the invention provides methods for ¹⁸F-radiolabeling of SiFAs by isotopic exchange (see, e.g. Example 1 below). The novel class of heteroaromatic SiFAs described herein can be labeled with the PET isotope ¹⁸F on various platforms. In one embodiment, the isotopic exchange is performed on various platforms including a commercial radiosynthesizer (ELYXIS, Sofie Biosciences), an in-house developed microfluidic Teflon™-coated chip, and a manual procedure in a sealed glass vial.

Scheme 2 immediately below depicts an exemplary method of performing the ¹⁹F to ¹⁸F isotopic exchange. Accordingly, a precursor ¹⁹F-SiFA compound of the current invention can be exchanged with an ¹⁸F-fluoride, to afford an ¹⁸F-compound of the current invention.

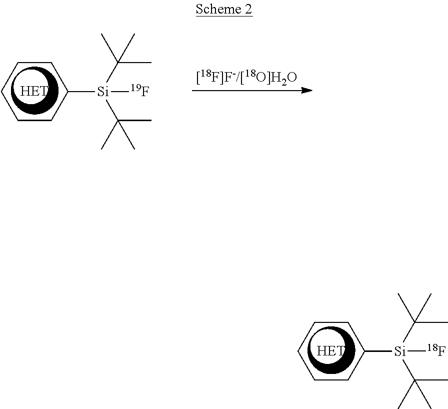 [SMILES: CC(C)(C)[Si]([19F])(C1CCCCC1)C(C)(C)C ; CC(C)(C)[Si]([18F])(C1CCCCC1)C(C)(C)C]

Purification of the labeled compound can be performed using any method known in the art. In a non-limiting example, purification of the final labeling product is achieved by a cartridge purification (C₁₈ or alumina).

As discussed in Example 1 below, the methods of making a heteroaromatic silicon-fluoride compound comprising a [¹⁸F] atom can comprise a number of steps such as disposing a [¹⁸F]fluoride donor compound within a cartridge comprising a quaternary methyl ammonium so that a [¹⁸F] tetraethyl ammonium fluoride compound is formed; and then eluting the [¹⁸F] tetraethyl ammonium fluoride compound from the cartridge with a solution comprising Tetraethylammonium bicarbonate at a concentration less than 50 umol. In these methods, the eluted [¹⁸F] tetraethyl ammonium fluoride compound is then dried and combined with a heteroaromatic silicon-fluoride compound precursor/¹⁸F-acceptor (e.g. a compound disclosed herein and comprising an [¹⁹F] atom that is exchanged with [¹⁸F] in this method) with the dry [¹⁸F] tetraethyl ammonium fluoride compound in an organic solvent (e.g. acetonitrile) so that the heteroaromatic silicon-fluoride acceptor compound and the dry [¹⁸F]tetraethyl ammonium fluoride compound exchange F isotopes; and then quenching the isotope-exchange reaction with water, so that the heteroaromatic silicon-fluoride compound comprising the ¹⁸F atom is made. In certain embodiments of these methods, the [¹⁸F] tetraethyl ammonium fluoride compound is eluted from the cartridge with a solution comprising Tetraethylammonium bicarbonate at concentrations less than 50 μmol, 40 μmol, 30 μmol or 20 μmol, for example between 5 μmol and 15 μmol. Typically these methods are performed at room temperature and/or obtain a radiochemical conversion of at least 80% or at least 90%. In certain embodiments of these isotope exchange methods, the heteroaromatic silicon-fluoride acceptor compound is combined with the dry [¹⁸F] tetraethyl ammonium fluoride compound in the organic solvent for not more than 10 minutes.

### Illustrative Compound Structures

In one aspect, the invention provides a compound of Formula 1:

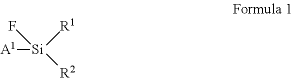

wherein in Formula 1,
F is selected from the group consisting of ¹⁹F and ¹⁸F; A¹ is a monocyclic or bicyclic heteroaryl ring optionally substituted with 0-4 R^{a} groups; R^{a} is selected, at each independent occurrence, from the group consisting of null, H, F, Cl, Br, I, CN, NO₂, OR^{c}OC(═O)R^{c}, OC(═O)OR^{c}, OC(═O)NR^{c}R^{d}, CR^{c}R^{d}, COR^{c}, C(═O)R^{c}c, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, or independent R^{a} groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R^{c}, R^{d} or R^{e} can optionally be joined to form additional rings; and R¹ and R² are each independently an alkyl group.

In another aspect, the invention provides a compound of Formula 2:

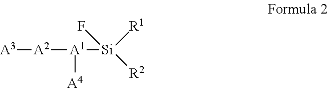

wherein in Formula 2, F is selected from the group consisting of ¹⁹F and ¹⁸F; A1 is a monocyclic or bicyclic heteroaryl ring optionally substituted with 0-4 R^{a} groups; A² is a linker; A³ is a moiety capable of chemical conjugation or bioconjugation; A⁴ is a moiety comprising a polar auxiliary moiety that may optionally contain a charge; R^{a} is selected, at each independent occurrence, from the group consisting of null, H, F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}, OC(═O)R^{c}, OC(═O)OR^{c}, OC(═O)NR^{c}R^{d}, CWRd, COR, C(═O)R, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(.circleincircle.O)₂R^{c}, and S(═O)₂NR^{c}R^{d}, or independent R^{a} groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R, R^{d} or R^{e} can optionally be joined to form additional rings; and R¹ and R² are each independently an alkyl group.

In another aspect, the invention provides a compound of Formula 3:

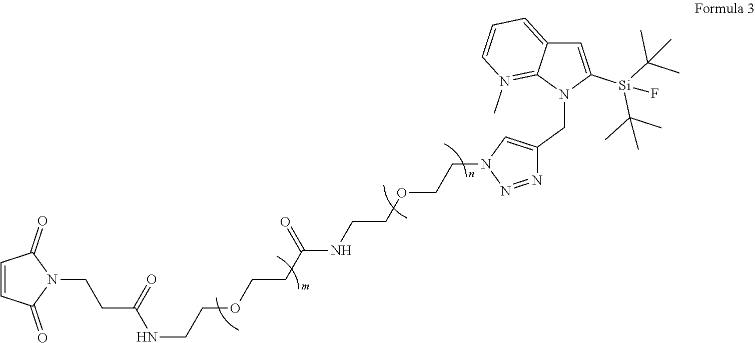

wherein in Formula 3, F is selected from the group consisting of ¹⁹F and ¹⁸F; and m and n are each independently an integer selected from the group consisting of 0, 1, 2, 3, 4, 5, and 6.

In another aspect, the invention provides a compound of Formula 4:

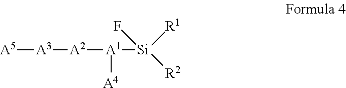

wherein in Formula 4, F is selected from the group consisting of ¹⁹F and ¹⁸F; A¹ is a monocyclic or bicyclic heteroaryl ring optionally substituted with 0-4 R^{a} groups; A² is a linker; A³ is a moiety capable of chemical conjugation or bioconjugation; A⁴ is a moiety comprising a polar auxiliary moiety that may optionally contain a charge; A⁵ is a moiety comprising a disease targeting molecule or biomolecule; R^{a} is selected, at each independent occurrence, from the group consisting of null, H, F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, 3, 4, or 5 substituents independently selected from F, Cl, Br, I, CN, NO₂, OR^{c}, OC(═O)R^{c}, OC(═O)OR^{c}, OC(═O)NR^{c}R^{d}, CR^{c}R^{d}, COR^{c}, C(═O)R^{c}, C(═O)NR^{c}R^{d}, C(═O)OR^{c}, NR^{c}R^{d}, NR^{c}C(═O)R^{d}, NR^{c}C(═O)OR^{d}, NR^{c}C(═O)NR^{d}R^{e}, NR^{c}S(═O)₂R^{d}, NR^{c}S(═O)₂NR^{d}R^{e}, SR^{c}, S(═O)R^{c}, S(═O)₂R^{c}, and S(═O)₂NR^{c}R^{d}, or independent R^{a} groups can optionally be joined to form additional rings; R^{c}, R^{d} and R^{e} are selected, at each independent occurrence, from the group consisting of H, and optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, and any of R^{c}, R^{d} or R^{e} can optionally be joined to form additional rings; and R¹ and R² are each independently an alkyl group.

In one embodiment, the heteroaromatic ring A¹ is selected from the group consisting of indole, azaindole, 7-azaindole, benzothiophene, furan, pyrrole, pyrazole, imidazole, and pyridine. In one embodiment, R¹ and R² each independently selected from the group consisting of methyl, ethyl, propyl, isopropyl, and tert-butyl. In one embodiment, R¹ and R² are tert-butyl groups. In one embodiment, the heteroaromatic ring A¹ is selected from the group consisting of indole, 7-azaindole, benzothiophene, furan, pyrrole, pyrazole, imidazole, and pyridine, and R¹ and R² are tert-butyl groups. In one embodiment, the linker A² includes an unsubstituted alkyl. In one embodiment, the linker A² includes an unsubstituted polyethylene glycol (PEG). In one embodiment, the linker A² includes a PEG4 linker. In one embodiment, the linker A² includes a PEG6 linker. In one embodiment, the linker A² includes a disubstituted triazole. In one embodiment, A³ is selected from the group consisting of an activated ester such as succinimide, an N-hydroxysuccinimide (NHS) ester, a maleimide, an amide, and a maleimide-thiol adduct. In one embodiment, a PEG-spacer is added for additional polarity. In one embodiment, A⁴ is a carboxylic acid. In one embodiment, A⁵ is an engineered antibody fragment. In one embodiment, A⁵ is an anti-PSCA A2 cys-diabody.

Exemplary embodiments of certain heteroaromatic SiFA elements of the invention are highlighted in Tables 2 and 3 of US Patent Publication 20180346491.

An illustrative embodiment of the invention includes the ¹⁸F radiolabeling of functionalized benzothiophene SiFA (NMK-BT-1) on a commercial radiosynthesizer (ELYXIS, Sofie Biosciences). In this embodiment, the HO—C═O (carboxylic acid) group functions as a handle moiety.

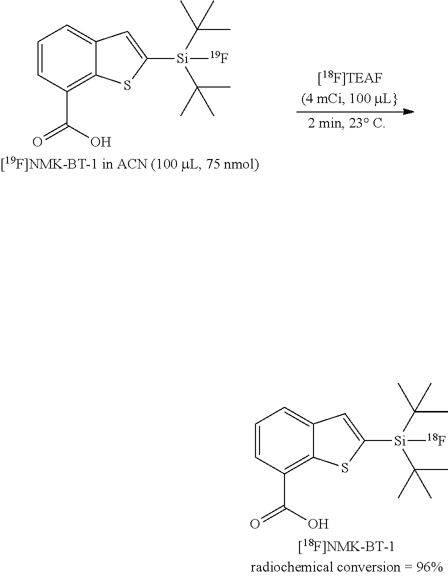 [SMILES: CC(C)(C)[Si]([19F])(c1cc2cccc(C(=O)O)c2s1)C(C)(C)C ; CC(C)(C)[Si]([18F])(c1cc2cccc(C(=O)O)c2s1)C(C)(C)C]

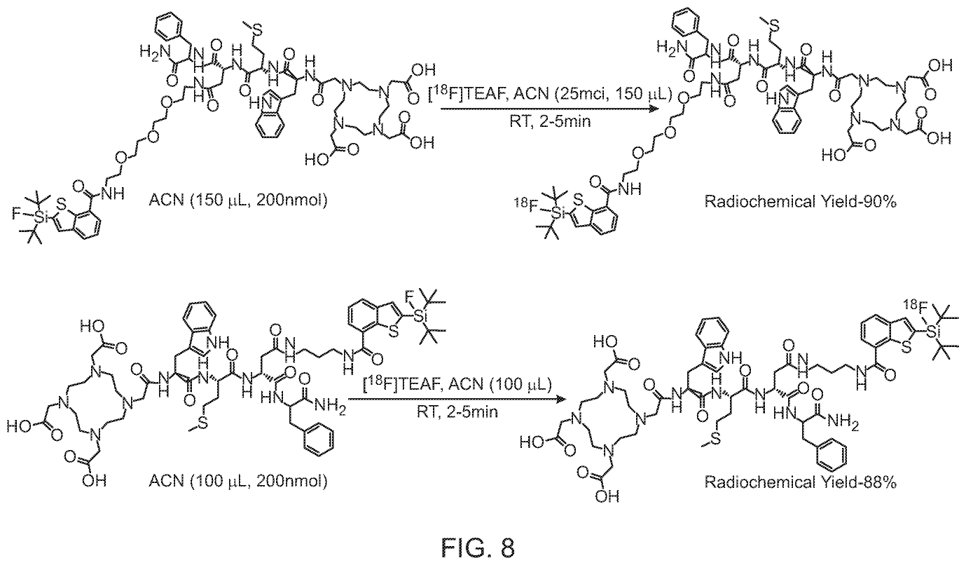

In illustrative working embodiments disclosed herein, a benzothiophene structure was used in positron emission tomography (PET) probe development and subsequent imaging studies. The benzothiophene was attached to a peptide via a PEG linker and a polar group to improve biodistribution. This example is the first actual demonstration that the benzothiophene prosthetic group is useful as Heteroaromatic silicon-fluoride-acceptor compounds as described above. The precursor compound was radiolabeled with fluorine-18 via isotopic exchange, purified with C18 cartridge and reformulated for in vivo imaging. Two examples are shown in the schematics of FIG. 8.

### Preparation of the Compounds of the Invention

The compounds of the present invention may be synthesized using techniques well-known in the art of organic synthesis (see, e.g., US Patent Publication 2018/0346491). The starting materials and intermediates required for the synthesis may be obtained from commercial sources or synthesized according to methods known to those skilled in the art. The following examples illustrate non-limiting embodiments of the invention.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, sites on, for example, the heteroaromatic or aromatic ring portion of compounds of the invention are susceptible to various metabolic reactions. Incorporation of appropriate substituents on the heteroaromatic or aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

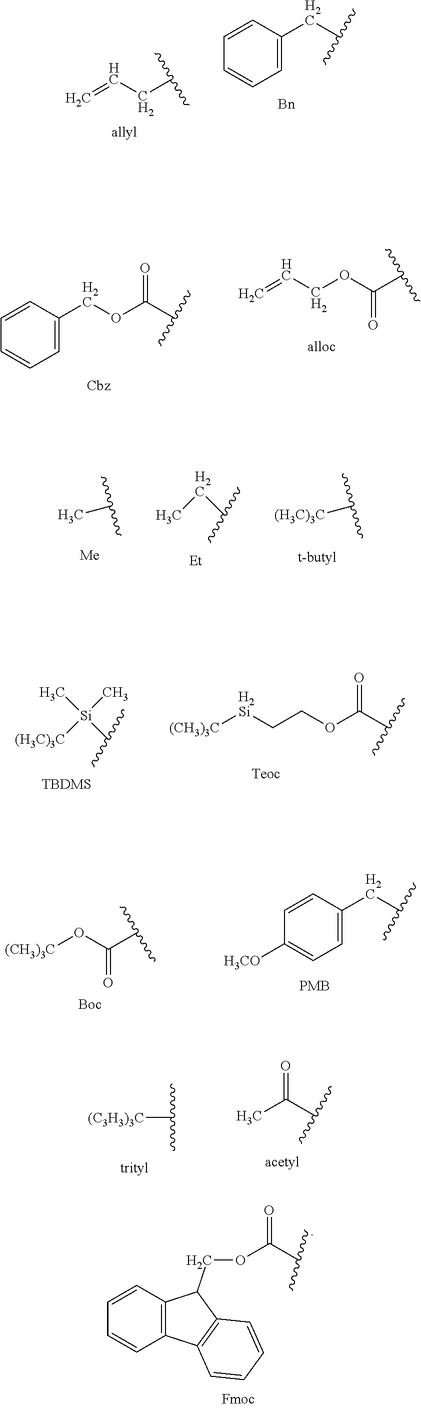

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

In one embodiment, the invention provides a method of synthesis of heteroaromatic Silicon-Fluoride Acceptors (SiFAs). In one embodiment, the precursors for SiFAs are synthetically accessible by a methodology using potassium tert-butoxide as a catalyst for the silylation of C—H bonds in aromatic heterocycles, methodology described by Toutov et al., Nature, 2015, 518:80-84, which is incorporated herein in its entirety.

Scheme 1 immediately below depicts an exemplary method for the synthesis of SiFAs. Accordingly, a heteroaromatic compound can be first treated with a catalytic amount of potassium tert-butoxide, and then reacted with di-tert-butyl silane, to afford an intermediate heteroarylsilane. The intermediate is thereafter reacted with potassium fluoride in the presence of a crown ether, to afford a ¹⁹F-SiFA compound of the current invention.

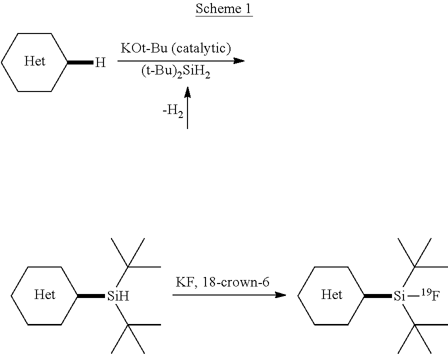

Compounds described herein include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed. In one embodiment, the compounds described herein are labeled by other means, including, but not limited to, the use of chromophores or fluorescent moieties, bioluminescent labels, or chemiluminescent labels.

### Kits of the Invention

The present invention encompasses various kits for ¹⁸F-labeling of heteroaromatic SiFAs, the kit comprising a heteroaromatic SiFA, an ¹⁸F-labeling reagent, and an instructional materials which describe use of the kit to perform the methods of the invention. These instructions simply embody the methods and examples provided herein. Although model kits are described below, the contents of other useful kits will be apparent to the skilled artisan in light of the present disclosure. Each of these kits is contemplated within the present invention. A kit is envisaged for each embodiment of the present invention.

The heteroaromatic SiFA of the present kit essentially includes the molecular elements/architectures disclosed elsewhere herein. The heteroaromatic SiFA can comprise a monocyclic or bicyclic heteroaryl ring optionally substituted, a linker, a moiety capable of chemical conjugation or bioconjugation, a moiety comprising a polar auxiliary moiety that may optionally contain a charge, and a moiety comprising a disease targeting molecule or biomolecule. The ¹⁸F-labeling reagent can comprise [¹⁸F]F⁻ from the cyclotron.

The kits of the present invention can further comprise additional reagents disclosed herein, such as plates and dishes used in the methods of the present invention, buffers, solutions and the like, as well as an applicator or other implements for performing the methods of the present invention. The kits of the present invention further comprise an instructional material. In one embodiment the kit comprises micropipettes, vials, a Teflon™-coated glass chip, a heater, and an alumina or other suitable purification cartridge.

An illustrative embodiment of invention is a kit for ¹⁸F-labeling of a compound disclosed herein, the kit comprising a compound disclosed herein wherein F is ¹⁹F. The kit further includes an ¹⁸F isotopic exchange reagent, and an instruction manual for the use thereof. In illustrative embodiments of the invention, the kit includes a container comprising a nonpolar solution comprising Tetraethylammonium bicarbonate at a concentration below 50 μmol, for example, at a concentration from about 1 μmol to about 10, 20, 30 or 40 μmol (e.g. about 10 μmol as discussed in Example 1 below).

### Methods for Imaging a Biological Target by Positron Emission Tomography

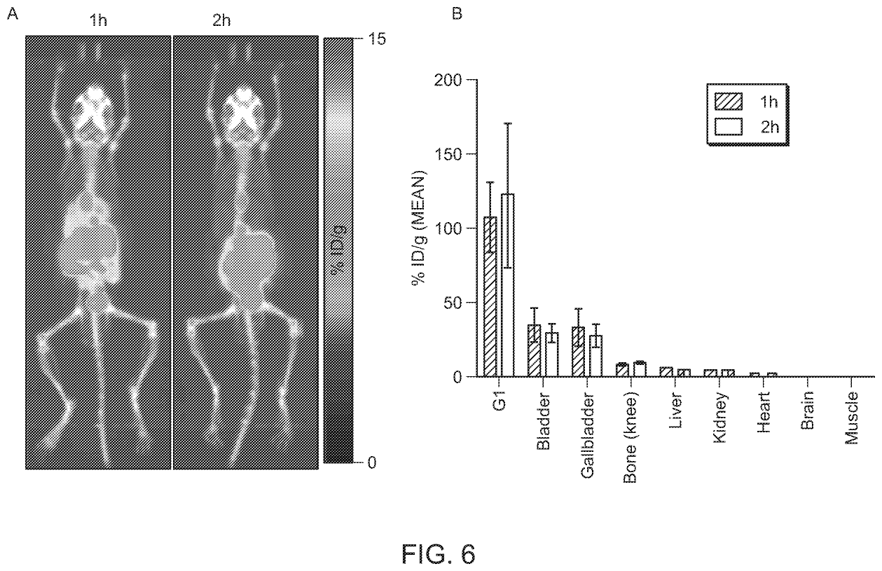

Embodiments of the invention include methods for imaging a biological target by positron emission tomography. Typically these methods comprise introducing into the target an imaging agent comprising: a composition disclosed herein, wherein F is ¹⁸F; and then imaging the target by a positron emission tomography process such that the biological target is imaged by positron emission tomography. In certain embodiments of the invention, the biological target comprises a human organ, human tissue or human cancer cells. These methods of the invention can include a variety of conventional PET steps and/or use a variety of conventional PET reagents, for example, those discussed in “Positron Emission Tomography: Clinical Practice” by Peter E Valk; Dominique Delbeke; and Dale L Bailey. Data from a working example of this methodology is shown in FIG. 6.

In one embodiment, the imaging agent includes a compound disclosed herein, and a ligand for the target. In one embodiment, F in the compounds disclosed herein is ¹⁸F. In another embodiment, the ligand is a disease targeting molecule or biomolecule. In another embodiment, the ligand is a peptide. In another embodiment, the ligand is a protein. In another embodiment, the ligand is an enzyme. In another embodiment, the ligand is an antibody. In another embodiment, the ligand is a small molecule.

In another embodiment, the imaging agent is obtained by site-selective chemical conjugation of the ligand with the compound. In one embodiment, conjugation of the ligand occurs via a thiol group. In another embodiment, conjugation of the compound occurs via a N-hydroxysuccinimide (NHS) ester, a maleimide, or a click chemistry adduct.

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental example (for other exemplary methods and materials useful to make and practice embodiments of the invention, see also US Patent Publication 20180346491 which is incorporated by reference herein). The example is provided for purposes of illustration only, and are not intended to be limiting unless otherwise specified. Thus, the invention should in no way be construed as being limited to the following example, but rather, should be construed to encompass any and all variations which become evident as a result of the teaching provided herein.

### Example 1: Rapid One-Step ¹⁸F-Labeling of Peptides Via Heteroaromatic Silicon-2 Fluoride Acceptors

As discussed for example in Narayanam et al., Org. Lett. 2020, 22, 3, 804-808 (2020), positron emission tomography (PET) molecular imaging combines exquisite selectivity with remarkable sensitivity to provide high-resolution imaging of an expressed biomarker throughout all tissues of the body. Peptide-based probes have extraordinary potential as tools for cancer diagnosis due to their recognition of protein overexpression in many cancer types, which can be exploited for targeting purposes. The distinguishing characteristics of fluorine-18 such as favorable half-life (109.8 min), high positron efficiency (97%), and broad availability continue to make it the preferred radionuclide for clinical PET. To circumvent lengthy multistep reaction sequences for labeling peptides, approaches that employ fluorophilic elements such as Si (1-6), B (1,3,7-9), Al (9-11), and P (12,13) are becoming attractive options for the rapid production of kinetically stable fluorine-18-labeled PET tracers (14).

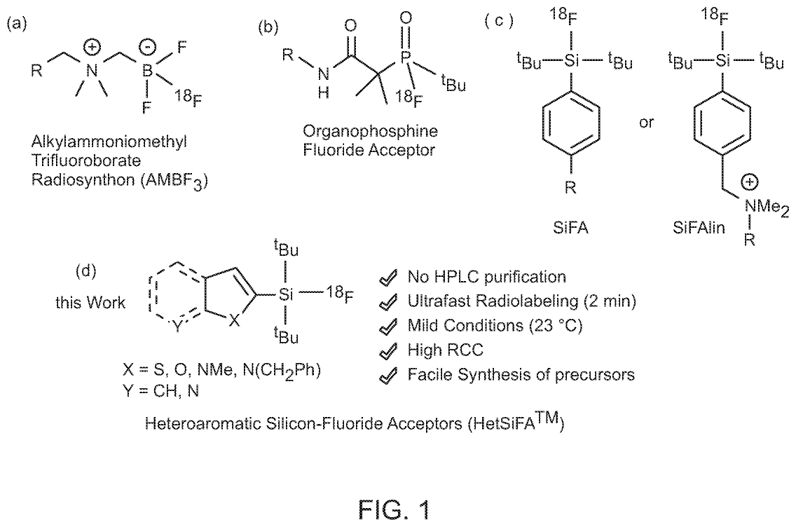

Driven by the demand for simple, rapid, and mild ¹⁸F-labeling protocols, isotope-exchange methods are becoming increasingly more popular for applications in PET research. Isotope exchange is generally conducted under mild conditions, and tracers can be purified via a simple C-18 cartridge-based system, eliminating the need for time-consuming high-performance liquid chromatography (HPLC) purification. Much has been learned in the past decade regarding ¹⁸F-labeling via isotope exchange, particularly by groups that challenged the paradigm to obtain high-molar-activity radio-tracers (15). Schirrmacher and Jurkschat et al. introduced silicon-fluoride acceptor (SiFA) systems and evaluated the ¹⁸F—Si hydrolytic stability in vivo (FIG. 1) (16). Stability studies of structurally hindered organofluorosilanes, including [¹⁸F]di-tert-butylphenyl fluorosilane, revealed the critical importance of steric hindrance around the silicon atom for practical utility as PET radiosynthons, which were first proposed by Rosenthal et al. (17). More recently, an organophosphine fluoride acceptor was reported to provide labeled peptides in moderate radiochemical yield using aqueous [¹⁸F]fluoride at room temperature (FIG. 1) (13).

In parallel, the exquisite strength of the fluorine-boron bond (732 kJ mol 49⁻¹) led to the investigation of organo-trifluoroborates as substrates for ¹⁸F-labeling approaches (8). Perrin reported that organosilicon and organoboron bio-conjugates undergo rapid, one-step fluorination at pH 4-7 in aqueous solvent to afford tetrafluorosilicates and trifluorobo-rates, respectively (1). Subsequent work from Perrin's group has pioneered synthetic access to [¹⁸F]-aryltrifluoroborates, including a [¹⁸F]-heteroaryltrifluoroborate (18, 19). Whereas various silicon- and boron-based compounds have been developed, persistent optimization to increase the fluorine-bond stability and decrease the lipophilicity has refined initial methods and led to new generations of radiosynthons bearing quaternary ammonium cations, namely, SiFAlin and AMBF3 (FIG. 1) (15, 20-22). Despite doubt, the fluorine-18 labeling of both organosilicon- and organoboron-based synthons via isotope exchange have produced high-molar-activity radiotracers within 30 min (15, 21, 23-25).

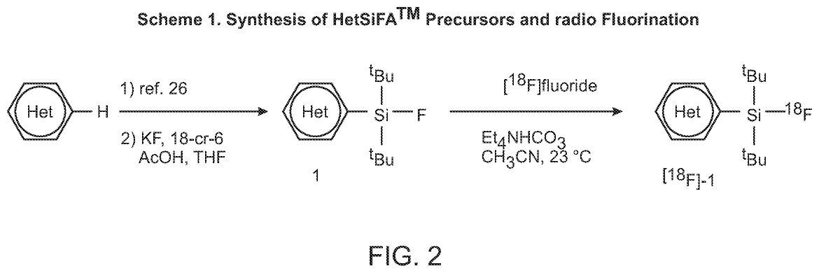

We sought to develop an isotope-exchange methodology that would incorporate heteroatoms such as oxygen, nitrogen, and sulfur into the SiFA systems (FIG. 1). We hypothesized the heteroaromatic silicon-fluoride acceptor systems would provide appealing alternative SiFA substrates, with decreased lipophilicity and facile construction compared with phenyl SiFA. The synthesis of phenyl SiFAs requires harsh conditions, generally employing the metal-halogen exchange of the aryl bromide with tBuLi, followed by nucleophilic substitution with di-tert-butylchlorosilane. Inspired by the recently reported potassium tert-butoxide-catalyzed C—H silylation of aromatic heterocycles (26), a mild approach for the synthesis of diverse Heteroaromatic silicon fluoride acceptor precursors was pursued, and utility toward radiosynthons for PET imaging was evaluated. To preserve the silicon-fluorine bond and retain hydrolytic stability, tert-butyl groups around the silicon atom were preserved in the heteroaromatic analogues. Scheme 1 in FIG. 2 depicts the synthetic pathway to afford heteroarylsilanes in good yield from commercial heteroarenes, which subsequently underwent fluorination with potassium fluoride and 18-crown-6 (1,4,7,10,13,16-hexaoxacyclooctadecane) in the presence of acetic acid to afford Heteroaromatic silicon fluoride acceptor precursors 1. The rapid ¹⁸F-fluorination of Heteroaromatic silicon fluoride acceptor precursors at room temperature delivered [¹⁸F]-1 in one step and in high radiochemical yield (Scheme 1 in FIG. 2).

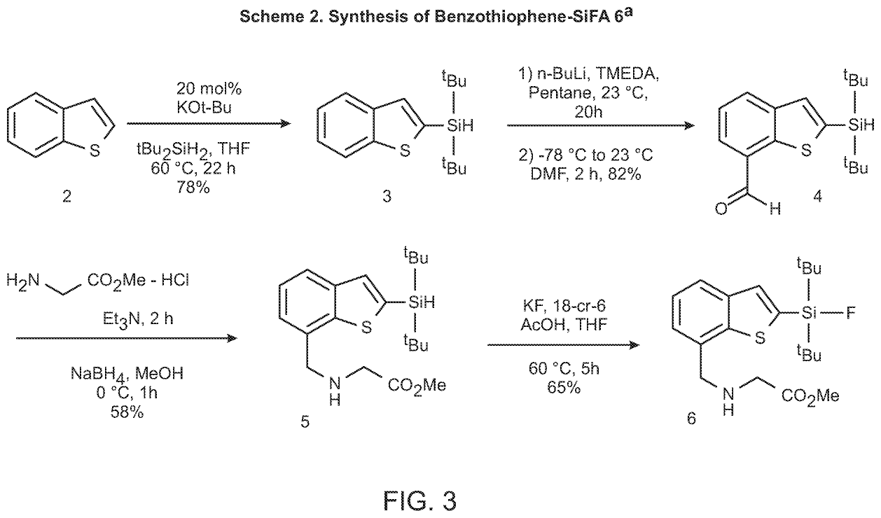

The isotope exchange of Heteroaromatic silicon fluoride acceptors with [¹⁸F]fluoride was first examined with compound 6 under conditions analogous to those demonstrated for phenyl SiFA systems (4). Glycine-functionalized Heteroaromatic silicon fluoride acceptor 6 was synthesized in four steps starting from commercially available benzothiophene 2 (Scheme 2 in FIG. 3). Potassium-catalyzed silylation provided the di-tert-butyl benozothiophenyl silane 3 in 78% yield in one step. The selective formylation at C7 of the benzothiophene (27) followed by the reductive amination of the resulting aldehyde generated benzothiophenyl intermediate 5 in good yield. Fluorination using standard conditions afforded Heteroaromatic silicon fluoride acceptor 6.

In a typical radiolabeling experiment, aqueous [¹⁸F]fluoride is pushed through a quaternary methylammonium (QMA) cartridge and trapped. Residual water is removed via a stream of nitrogen passed through the QMA. The trapped [¹⁸F]-fluoride is eluted with a solution of Et4NHCO3 in acetonitrile/water (4:1) and azeotropically dried to afford dry tetraethy-lammonium fluoride ([¹⁸F]TEAF). A low precursor mass is critical to obtain high-molar-activity radiotracers via isotope-exchange methodologies. As such, the initial experiments examined the sensitivity of this reaction to the stoichiometry of Heteroaromatic silicon fluoride acceptor 6 (Table 1 herein and Table S1 below). With a 150 nmol precursor at 23° C. in CH3CN, [¹⁸F]-6 was obtained in 95±2% radiochemical conversion (RCC) after 2 min, as determined by radio-thin layer chromatography (radio-TLC) (Table 1, entry 1). Comparable RCC was obtained using a 100 nmol precursor, and with a 50 nmol precursor under the same conditions, [¹⁸F]-6 was provided in 87±2% RCC (Table 1, entry 9).

Whereas short reaction times are highly desirable for radiofluorination, we noted that extending the reaction time from 2 to 30 min caused a drop in the RCC from 95 to 77% (Table 1, entries 1-4) using a 150 nmol precursor under standard conditions. A similar trend in RCC was obtained in all initial experiments, independent of the precursor amounts (Table 1, entries 1-12). We hypothesized that the high concentration of Et4NHCO3 (50 μmol) present in the reaction mixture caused rapid hydrolysis of the ¹⁸F—Si bond. To minimize hydrolysis, the Et4NHCO3 used for [¹⁸F]fluoride elution was lowered to 5.75 μmol, and the stability of [¹⁸F]-6 was examined over time.

Under low base conditions, we were encouraged to see that radiofluorination proceeded in 94±1% RCC, with stable and, in some cases, a slightly higher conversion after 30 min (Table 1 herein, entries 1-15, far right column, and Table S2 below). Significantly lowering the stoichiometry to a 10 nmol precursor under the low base conditions resulted in 83±2% RCC after 2 min (Table 1, entry 13, far right column). Of note, a reduction in the amount of Et4NHCO3 overcame the hydrolysis of the ¹⁸F—Si bond but also resulted in lower elution efficiencies of [¹⁸F]fluoride. To optimize the [¹⁸F]fluoride elution, additional conditions were explored, and the amounts of Et4NHCO3 as well as the elution solvents were screened (Table S3 below). An acceptable elution efficiency was obtained with ˜10 μmol Et4NHCO3 to afford high RCC and provide stable ¹⁸F-labeled Heteroaromatic silicon fluoride acceptor products (Tables S4 and S5 below).

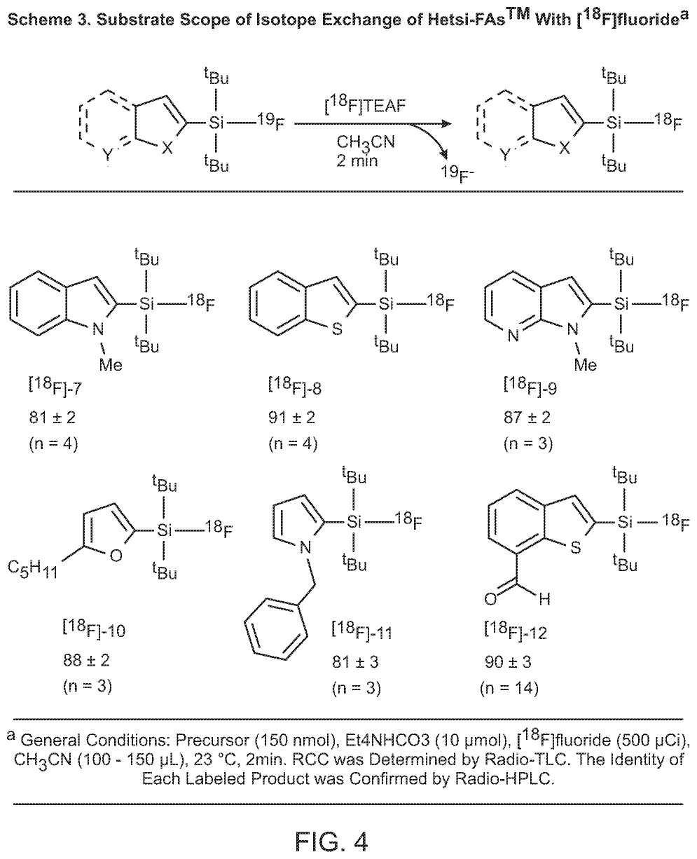

The optimized reaction conditions were applied to a series of diverse Heteroaromatic silicon fluoride acceptor radiosynthons, prepared via potassium tert-butoxide-catalyzed C—H silylation, as previously reported (Scheme 3 in FIG. 4; see also the Supporting Information below) (26). Precursors were subjected to a solution of dry [¹⁸F]TEAF (500 μCi) in acetonitrile at 23° C. to enable ¹⁸F-¹⁹F isotope exchange. The reaction was quenched with water, and an aliquot of the crude mixture was analyzed by radio-TLC and radio-HPLC to confirm the identity of the labeled Heteroaromatic silicon fluoride acceptors. High ¹⁸F incorporation was successfully achieved within 2 min in all cases, and >90% RCCs were obtained for benzothiophene-based constructs (Scheme 3 in FIG. 4). This methodology is compatible with heterocycles such as indole (7), benzothiophene (8, 12), azaindole (9), furan (10), and pyrrole (11). The conversions are comparable and, in some cases, considerably higher than analogous isotope-exchange methodologies. We opted to conduct the isotope exchange on a 150 nmol precursor; however, the stoichiometry of the Heteroaromatic silicon fluoride acceptor could be as low as 10 nmol with a marginal decrease in the RCC (Table 1 herein and Table S5 below).

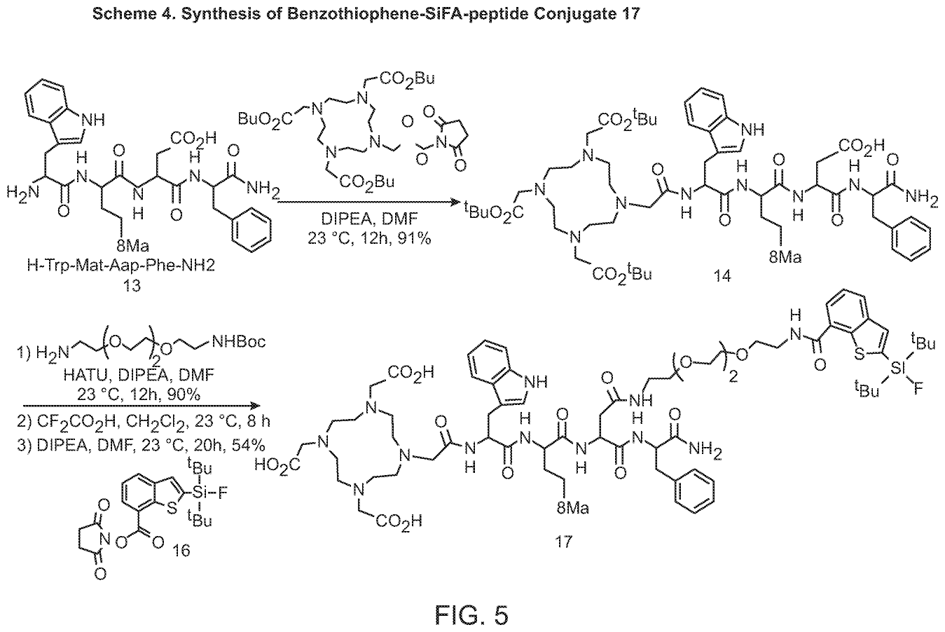

We next focused on applying the benzothiophene-SiFA toward peptide-labeling applications. For facile conjugation to peptides, N-hydroxysuccinimide ester 16 was synthesized in five steps starting from the commercially available benzothiophene 2 (Scheme S1 below). Similar functionalization could be applied toward the heteroaromatic silanes in Scheme 3 in FIG. 4; to afford radiosynthons of diverse heteroaromatic systems. A proof-of-concept study to evaluate the in vivo stability of peptide-based Heteroaromatic silicon fluoride acceptor tracers was conducted using a commercial peptide derived from the hormone cholecystokinin, cholecystokinin tetrapeptide (CCK-4). CCK-4 is a small peptide fragment with the sequence H-Trp-Met-Asp-Phe-NH2 (Scheme 4 in FIG. 5).

Properties such as lipophilicity and charge strongly influence the pharmacokinetics of radiolabeled peptides and their metabolites (28, 29). Modulation with hydrophilic and anionic chemical moieties has revealed a strong influence on tracer biodistribution and radiometabolite clearance (20, 30). Recently, the incorporation of a metal-free DOTA appendage demonstrated the ability to redirect clearance to the kidneys over the liver and significantly reduce gastrointestinal (GI) tract retention for radiolabeled peptides (31). In addition to the influence on renal clearance, the DOTA appendage provides further utility toward the chelation of a therapeutic radioisotope for targeted radionuclide therapy. Radiopharmaceuticals that offer multi-isotope labeling for theranostic applications present valuable opportunities for a unique class of tunable tracers, allowing for the optimal selection of radioisotope for a given circumstance (32).

To decrease the tracer lipophilicity and improve the biodistribution profile, a metal-free DOTA auxiliary was appended to CCK-4 13 to afford peptide-DOTA conjugate 14 in excellent yield (Scheme 4 in FIG. 5). Functionalization with a PEGylated amine was achieved under standard coupling conditions followed by acid deprotection and coupling to N-hydroxysuccinimide ester 16 to afford conjugate 17 (Scheme 4 in FIG. 5). Using the previously optimized reaction conditions, radiofluorination of conjugate 17 proceeded efficiently in 94±1% RCC (n=3) (Table S6 below). The crude reaction was quenched and passed through a C18 Sep-Pak. After a water wash and reformulation, analysis by radio-TLC and radio-HPLC confirmed the isotope exchange of peptide conjugate 17 to yield [¹⁸F]-17 in 58±6% isolated radiochemical yield (RCY) (n=3) and >99% radiochemical purity. The molar activity of [¹⁸F]-17 was 0.032 Ci/μmol, on par with previous reports for isotope-exchange reactions with low starting activity (33). Following literature precedent, we expect to achieve higher molar activity when scaling up to larger starting radioactivity levels, for example, >1 Ci (34).

A loss of activity occurred during the reformulation process, which was unoptimized. An investigation of solvent-exchange cartridges will be performed in future studies to avoid these losses. The total reaction time from [¹⁸F]fluoride to the formulated radiotracer was 32 min.

Micropositron emission tomography-computed tomography (microPET/CT) imaging studies were performed in normal mice to investigate the in vivo stability of the silicon-fluorine bond in the Heteroaromatic silicon fluoride acceptor peptide conjugate, [¹⁸F]-17. Reformulated [¹⁸F]-17 (2.2 MBq) was injected via the tail vein into C57BL/6 mice (n=3), and microPET/CT scans were acquired. MicroPET/CT images confirm the initial clearance of [¹⁸F]-17 via the kidneys, followed by the reabsorption of radiometabolites and the subsequent hepatobiliary clearance, resulting in high liver and gallbladder uptake as well as retention of activity in the GI tract (FIG. 6). Whereas [¹⁸F]-17 demonstrated rapid plasma clearance, residual activity in the liver and retention in the GI are likely due to the lipophilicity of radiometabolites. Alternatively, the GI activity may also be a result of this particular peptide, for which the full cholecystokinin construct plays important roles in stimulating digestion within the GI system.

FIG. 6 reveals some bone uptake (8% ID/g) that may be due to the specific bone uptake of [¹⁸F]-17 and its metabolites or to [¹⁸F]fluoride hydrolysis and release. Whereas additional studies are needed to verify the cause of bone uptake, a combination of variables, including the nature of this particular peptide, are likely to be contributing factors (20, 35). Alternative peptides will be explored in future studies with a judicious selection of conjugation sites to confirm the broad applicability of the Heteroaromatic silicon fluoride acceptor scaffold. Importantly, the relative tracer uptake compared with background (nontarget tissue, muscle, etc.) is the critical measurement for diagnostic PET imaging applications. The biodistribution of [¹⁸F]-17 reveals a low muscle uptake of 0.36% ID/g (Table S7 below), suggesting that target tissue would be clearly distinguishable among surrounding tissue (with the exception of the GI tract) with a high contrast and signal-to-noise ratio.

In summary, the method disclosed herein represents the first demonstration of SiFA-based heteroaromatic systems for radiofluorination, is compatible with multiple heterocycles, and provides a simple, late-stage ¹⁸F-labeling approach for peptide-based radio-pharmaceutical production. Additionally, this work compliments the repertoire of unique labeling approaches involving organosilicon-based radiosynthons, which have recently shown promise in the clinic (36, 37). Furthermore, microscale radiosynthesis platforms have enabled the production of PET tracers with consistently high molar activity (˜20 Ci/μmol) independent of starting radioactivity (34).

### Example 1 Supplementary Information

### 1. Materials and Methods

All reagents and chemicals were purchased from commercial sources and used without further purification. DOTA-mono-NHS tris (t-Bu ester) (product number B-270) was purchased from Macrocyclics. Gastrin Tetrapeptide (H-Trp-Met-Asp-Phe-NH₂) (cas no [1947-37-1]) was purchased from Bachem Americas (product number H-3110). All deuterated solvents were purchased from Cambridge Isotope Laboratories. Unless otherwise noted, reactions were carried out in oven-dried glassware under an atmosphere of argon using commercially available anhydrous solvents. Solvents used for extractions and chromatography were not anhydrous. Silicon oil bath was used as the heating source for all reactions. Reactions and chromatography fractions were analyzed by thin-layer chromatography (TLC) using Merck precoated silica gel 60 F254 glass plates (250 μm) and visualized by ultraviolet irradiation, 2,4-dinitrophenyl hydrazine or potassium permanganate stain or ninhydrin stain. Flash column chromatography was performed using E. Merck silica gel 60 (230-400 mesh) with compressed air and ethyl acetate and n-hexane were used as eluent solvents. NMR spectra were recorded on a Bruker ARX 400 (400 MHz for 1H; 100 MHz for 13C) spectrometer. Chemical shifts are reported in parts per million (ppm, δ) using the residual solvent peak as the reference. The coupling constants, J, are reported in Hertz (Hz), and the multiplicity identified as the following: br (broad), s (singlet), d (doublet), t (triplet), q (quartet), and m (multiplet). High-resolution electrospray mass spectrometry data was collected with a Waters LCT Premier XE time-of-flight instrument controlled by MassLynx 4.1 software. For some samples, high-resolution mass spectra were obtained on Thermo Scientific™ Exactive Mass Spectrometer with DART ID-CUBE. Samples were dissolved in methanol and infused using direct loop injection from a Waters Acquity UPLC into the Multi-Mode Ionization source. HPLC purifications were performed on a Knauer Smartline HPLC system with inline Knauer UV (210 or 254 nm) detector. Semi-preprative HPLC was performed using Phenomenex reverse-phase Luna column (10×250 mm, 5 μm) with a flow rate of 4 mL/min. Final purity of compounds was determined by analytical HPLC analysis performed with a Phenomenex reverse-phase Luna column (4.6×250 mm, 5 μm) with a flow rate of 1 mL/min. Compounds were identified by UV absorbance at 254 nm. All chromatograms were collected by a GinaStar (raytest USA, Inc.; Wilmington, N.C., USA) analog to digital converter and GinaStar software (raytest USA, Inc.).

### 2. Experimental Data

### 2.1 Synthesis and Characterization of Heteroarylsilanes

Heteroarylsilanes were synthesized according to literature procedure. See, e.g. Toutov et al., Nature 2015, 518, 80. A representative example is described below.

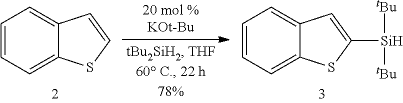

### benzo[b]thiophen-2-yldi-tert-butylsilane (3)

To a vial containing benzothiophene (134.2 mg, 1 mmol), potassium tert-butoxide (22.5 mg, 0.2 mmol) and di-tert-butylsilane (0.59 mL, 3.0 mmol) was added THF (1.0 mL) inside a glovebox. The vial was sealed, taken outside the glovebox and stirred at 60° C. for 22 h. The reaction mixture was concentrated in vacuo and the crude residue was purified by silica gel column chromatography eluting with 100% hexane to afford 3 (214.0 mg, 78%) as a white solid. 1H NMR (400 MHz, CDCl3) δ 7.92-7.87 (m, 1H), 7.86-7.82 (m, 1H), 7.58 (d, J=0.7 Hz, 1H), 7.39-7.30 (m, 2H), 4.09 (s, 1H), 1.11 (s, 18H). 13C NMR (100 MHz, CDCl3) δ 143.8, 140.7, 135.0, 133.8, 124.2, 124.0, 123.4, 122.0, 29.4, 19.0. HRMS (APCI) m/z calcd for C16H24FSSi [M]+276.1368, found 276.1364.

### 2.2 Synthesis of Glycine Ester HetSiFA 6

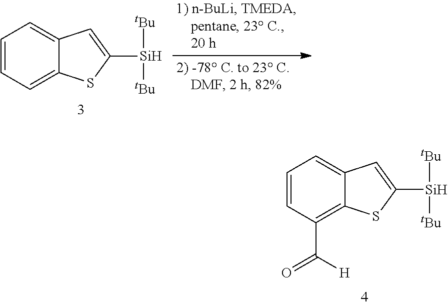

### 2-(di-tert-butylsilyl)benzo[b]thiophene-7-carbaldehyde (4)

To a flame dried round bottom flask benzothiophene 3 (304.2 mg, 1.1 mmol), TMEDA (0.25 mL, 1.65 mmol) and pentane (3 mL) was added under a steady stream of argon. n-Butyllithium (2.5 M in hexanes, 0.66 mL, 1.65 mmol) was added dropwise such that the internal temperature remained between 22 and 25° C. The S 20 mol % KOt-Bu tBu2SiH2, THF 60° C., 22 h 78% S SiH tBu tBu 2 3 S SiH 1) n-BuLi, TMEDA, pentane, 23° C., 20 h 2) −78° C. to 23° C. DMF, 2 h, 82% tBu tBu S SiH tBu tBu 3 O H 4 S5 reaction mixture was stirred at room temperature for 20 h. The solution was then cooled to −78° C. (dry ice/acetone bath) and N,N-dimethylformamide (0.32 mL, 4.4 mmol) was added dropwise such that the temperature was kept at −78° C. The resulting solution was allowed to stir at −78° C. for 1 h before it was brought to room temperature and stirred at 23° C. for additional 1 h. The dark colored reaction mixture was carefully quenched with saturated aqueous NH₄Cl (3 mL). The crude product was extracted with ethyl acetate (10 mL×2) and combined organics were washed with brine, dried over MgSO4 and concentrated under reduced pressure. The residue was purified by silica gel column chromatography eluting with 5% ethyl acetate in hexanes to afford aldehyde 4 (250 mg, 82%) as a yellow oil.

1H NMR (400 MHz, CDCl3): 1H NMR (400 MHz, CDCl3) δ 10.26 (s, 1H), 8.13 (dd, J=7.9, 1.1 Hz, 1H), 7.88 (dd, J=7.2, 1.1 Hz, 1H), 7.67 (s, 1H), 7.57 (dd, J=7.9, 7.2 Hz, 1H), 4.15 (s, 1H), 1.11 (s, 18H); 13C NMR (100 MHz, CDCl3) δ 191.3, 142.3, 141.0, 139.3, 132.6, 131.4, 130.6, 129.5, 124.0, 28.7, 19.0. HR-MS (APCI) m/z calcd for C17H25OSSi [M+H]+ 305.1395, found 305.1400.

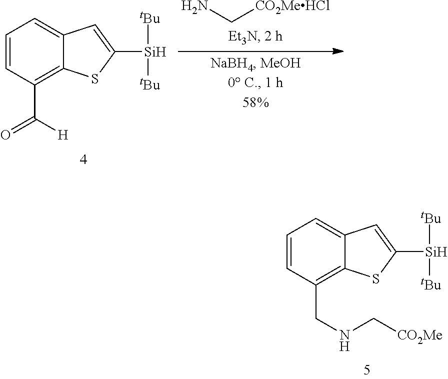

### Methyl ((2-(di-tert-butylsilyl)benzo[b]thiophen-7-yl)methyl)glycinate (5)

To a stirred solution of 4 (110 mg, 0.36 mmol) and glycine methyl ester hydrochloride (68 mg, 0.54 mmol) in methanol at 0° C. was added trimethylamine (0.075 mL, 0.54 mmol). The contents were stirred at 0° C. for 30 min before warming to room temperature and stirring for 1.5 h. The reaction mixture was cooled to 0° C. and sodium borohydride (27 mg. 0.72 mmol) was added in one portion and the reaction was stirred at 0° C. for 1 hr. The reaction was quenched with water (1 mL) and the crude product was extracted into ethyl acetate (25 mL) and washed with a saturated brine solution. The residue was purified on silica gel column chromatography eluting with 20% ethyl acetate in hexanes to obtain desired product 5 as colorless oil (79 mg, 58% yield).

1H NMR (400 MHz, CDCl3) δ 7.77 (dd, J=5.0, 4.0 Hz, 1H), 7.61 (s, 1H), 7.36-7.33 (m, 2H), 4.16 (s, 2H), 4.10 (s, 1H), 3.73 (s, 3H), 3.49 (s, 2H), 1.11 (s, 18H). 13C NMR (100 MHz, CDCl3) δ 172.7, 142.9, 141.3 134.8, 134.1, 132.9, 124.4, 123.4, 122.6, 52.1, 51.8, 49.8, 28.7, 19.1. HRMS (APCI) m/z calcd for C20H32NO2SSi [M+H]+ 378.1923, found 378.1911.

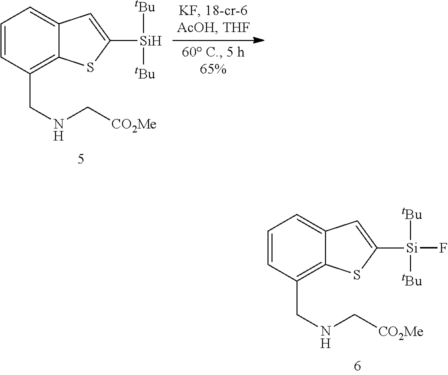

### Methyl ((2-(di-tert-butylfluorosilyl)benzo[b]thiophen-7-yl)methyl)glycinate (6)

To a stirred solution of 5 (64 mg, 0.17 mmol), potassium fluoride (15 mg, 0.26 mmol) and 18-crown-6 (67 mg, 0.26 mmol) was added THF (2 mL) and acetic acid (0.030 mL, 0.51 mmol). The contents were stirred at 60° C. for 5 h. The crude residue was filtered and concentrated under reduced pressure. The crude product was purified on silica gel column chromatography eluting with 30% ethyl acetate in hexanes to obtain 6 as colorless oil (44 mg, 65% yield).

1H NMR (400 MHz, CDCl3) δ 7.80 (dd, J=6.2, 2.9 Hz, 1H), 7.67 (s, 1H), 7.38 (dd, J=6.7, 4.2 Hz, 2H), 4.18 (s, 2H), 3.73 (s, 3H), 3.49 (s, 2H), 1.12 (d, J=1.0 Hz, 18H). 13C NMR (100 MHz, CDCl3) δ 172.6, 142.8, 141.0, 133.7 (d, J=4 Hz), 133.2, 132.9, 124.6, 123.8 (d, J=2.2 Hz), 122.9, 52.1, 51.8, 49.6 (d, J=3.5 Hz), 27.0, 20.3 (d, J=12.2 Hz). 19F NMR (376 MHz, CDCl3) δ−183.78. HRMS (APCI) m/z calcd for C20H31NFO2SSi [M+H]+ 396.1829, found 396.1815.

### 2.3 General Procedure for Fluorination of Heteroarylsilanes

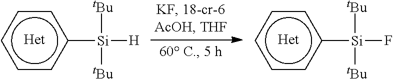

A round bottom flask containing the heteroarylsilane (0.22 mmol), potassium fluoride (0.33 mmol) and 18-crown-6-ether (0.33 mmol) was added THF (2 mL) and acetic acid (0.66 mmol) under S SiH tBu tBu NH CO2Me S Si tBu tBu NH CO2Me KF, 18-cr-6 F AcOH, THF 60° C., 5 h 5 65% 6 KF, 18-cr-6 AcOH, THF 60° C., 5 h Het Si tBu tBu Het Si F tBu tBu H S7 argon atmosphere. The reaction mixture was stirred at 60° C. for 5 h. After completion of the reaction, the crude mixture was filtered with dichloromethane and concentrated under reduced pressure. The residue was purified by silica gel column chromatography eluting with 10-15% ethyl acetate in hexanes to afford the desired heteroarylfluorosilanes.

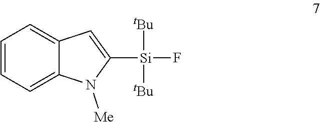 [SMILES: Cn1c([Si](F)(C(C)(C)C)C(C)(C)C)cc2ccccc21]

### 2-(di-tert-butylfluorosilyl)-1-methyl-1H-indole (7)

1H NMR (400 MHz, CDCl3) δ 7.67 (dt, J=8.0, 1.0 Hz, 1H), 7.40 (dd, J=8.4, 0.9 Hz, 1H), 7.29 (ddd, J=8.3, 6.9, 1.2 Hz, 1H), 7.14 (ddd, J=7.9, 6.9, 1.0 Hz, 1H), 6.84 (s, 1H), 3.95 (d, J=1.2 Hz, 3H), 1.14 (d, J=1.2 Hz, 18H). 13C NMR (100 MHz, CDCl3) δ 139.8, 128.2 (d, J=1.4 Hz), 122.3, 120.8, 119.4, 112.4, 109.4, 33.1, 27.2, 20.9 (d, J=12.1 Hz). 19F NMR (376 MHz, CDCl3) δ−181.04. HRMS (ESI) m/z calcd for C17H27FNSi [M+H]+ 292.1897, found 292.1895.

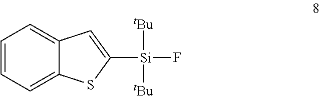 [SMILES: CC(C)(C)[Si](F)(c1cc2ccccc2s1)C(C)(C)C]

### benzo[b]thiophen-2-yldi-tert-butylfluorosilane (8)

1H NMR (400 MHz, CDCl3) δ 7.94-7.88 (m, 2H), 7.87 (s, 1H), 7.41-7.33 (m, 2H), 1.13 (d, J=1.1 Hz, 18H). 13C NMR (100 MHz, CDCl3) δ 143.6, 140.4, 133.3 (d, J=2.9 Hz), 133.1 (d, J=16.4 Hz), 124.7, 124.2, 123.8, 122.1, 27.0, 20.3 (d, J=12.0 Hz). 19F NMR (376 MHz, CDCl3) δ−183.95. HRMS (APCI) m/z calcd for C16H23FSSi [M+H]+ 294.1264, found 294.1263.

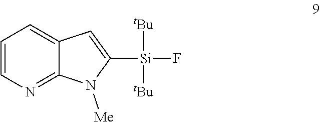 [SMILES: Cn1c([Si](F)(C(C)(C)C)C(C)(C)C)cc2cccnc21]

### 2-(di-tert-butylfluorosilyl)-1-methyl-1H-pyrrolo[2,3-b]pyridine (9)

1H NMR (400 MHz, CDCl3) δ 8.39 (dd, J=4.7, 1.5 Hz, 1H), 7.94 (dd, J=7.8, 1.4 Hz, 1H), 7.07 (dd, J=7.8, 4.7 Hz, 1H), 6.77 (s, 1H), 4.06 (d, J=2.2 Hz, 3H), 1.11 (d, J=1.0 Hz, 18H). 13C NMR (100 MHz, CDCl3) δ 149.0, 144.8, 142.4, 129.7, 126.7, 120.8, 116.0, 115.4, 110.6, 32.0, 22.0, 20.8 (d, J 11.3 Hz). 19F NMR (376 MHz, CDCl3) δ −181.45. HRMS (ESI) m/z calcd for C17H26FN2Si [M+H]+ 293.1849, found 293.1852.

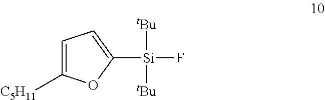

### di-tert-butylfluoro(5-pentylfuran-2-yl)silane (10)

1H NMR (400 MHz, CDCl3) δ 6.74 (d, J=3.1 Hz, 1H), 6.01 (d, J=3.1 Hz, 1H), 2.66 (t, J=7.5 Hz, 2H), 1.69-1.61 (m, 2H), 1.35-1.29 (m, 4H), 1.07 (d, J=1.1 Hz, 18H), 0.89 (t, J=7.0 Hz, 3H). 13C NMR (100 MHz, CDCl3) δ 161.4, 151.4 (d, J=23.3 Hz), 123.5, 104.7, 31.3, 28.0, 27.8, 26.8, 22.4, 20.0 (d, J=12.4 Hz), 13.9. 19F NMR (376 MHz, CDCl3) δ−187.21. HRMS (APCI) m/z calcd for C17H32FOSi [M+H]+ 299.2206, found 299.2212.

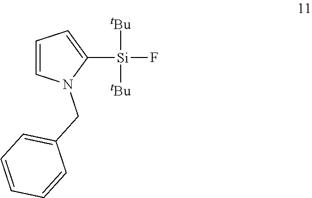 [SMILES: CC(C)(C)[Si](F)(c1cccn1Cc1ccccc1)C(C)(C)C]

### 1-benzyl-2-(di-tert-butylfluorosilyl)-1H-pyrrole (11)

1H NMR (400 MHz, CDCl3) δ 7.34-7.20 (m, 3H), 7.05 (dd, J=16.6, 7.0 Hz, 2H), 6.91 (dd, J=2.3, 1.4 Hz, 1H), 6.54 (td, J=4.0, 1.4 Hz, 1H), 6.30-6.24 (m, 1H), 5.28 (s, 2H), 1.02 (d, J=1.0 Hz, 18H). 13C NMR (101 MHz, CDCl3) δ 139.4, 128.4 (d, J=5.4 Hz), 127.2, 126.9, 126.7, 120.1 (d, J=6.0 Hz), 108.6, 53.4, 27.2, 20.9 (d, J=12.4 Hz). 19F NMR (376 MHz, CDCl3) δ−187.63. HRMS (APCI) m/z calcd for C19H29FNSi [M+H]+ 318.2053, found 318.2059.

### 2.4 Synthesis of N-hydroxysuccinimide ester HetSiFA 16

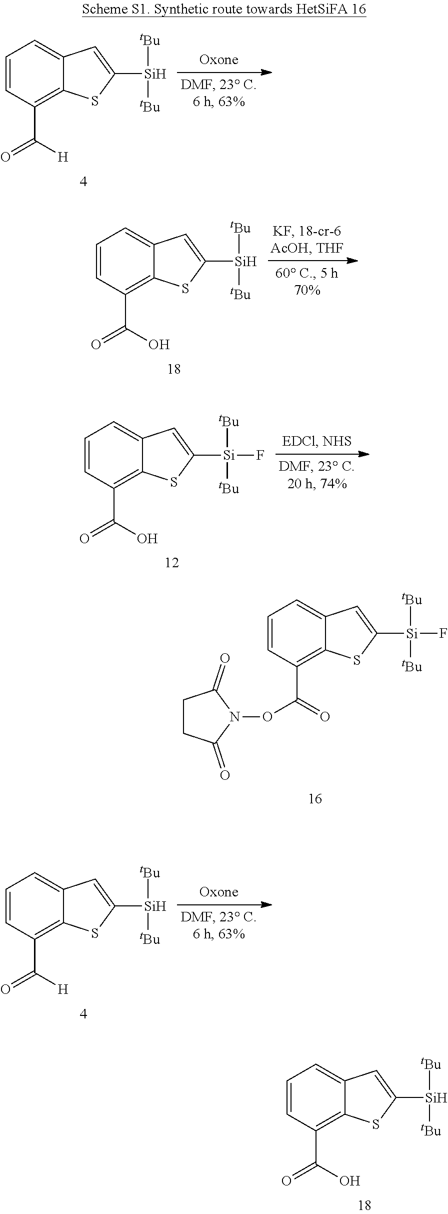

### 2-(di-tert-butylsilyl)benzo[b]thiophene-7-carboxylic acid (18)

See, e.g. Travis, et al., Org. Lett. 2003, 5, 1031. To a 10 mL round-bottom flask containing N,N-dimethylformamide (2 mL) and aldehyde 4 (128 mg, 0.42 mmol), was added Oxone@(135 mg, 1.05 mmol) in one portion and stirred at room temperature for 6 hrs. The reaction mixture was diluted with 1 M HCl (1 mL) and ethyl acetate (10 mL). The organic layer was washed with brine, dried over sodium sulfate and the solvent was removed under reduced pressure. The crude residue was purified by silica gel column chromatography eluting with hexanes:ethyl acetate (70:30, v/v) to obtain the desired acid 18 (84 mg, 63%) as an off-white solid.

1H NMR (400 MHz, CDCl3) δ 8.25 (dd, J=7.5, 1.2 Hz, 1H), 8.10 (dd, J=7.9, 1.2 Hz, 1H), 7.66 (s, 1H), 7.49 (t, J=7.7 Hz, 1H), 4.14 (s, 1H), 1.14 (s, 18H). 13C NMR (100 MHz, CDCl3) δ 171.5, 144.5, 142.3, 138.3, 133.1, 129.0, 128.0, 123.9, 123.2, 28.7, 19.1. HRMS (APCI) m/z calcd for C17H23O2SSi [M−H]−319.1194, found 319.1200.

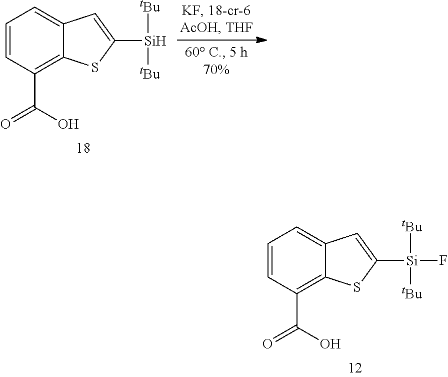

### 2-(di-tert-butylfluorosilyl)benzo[b]thiophene-7-carboxylic acid (12)

A round bottom flask containing carboxylic acid 18 (70 mg, 0.22 mmol), potassium fluoride (19 mg, 0.33 mmol) and 18-crown-6-ether (87 mg, 0.33 mmol) was added THF (2 mL) and acetic acid (0.04 mL, 0.66 mmol) under argon atmosphere. The reaction mixture was stirred at 60° C. for 5 hrs. After completion of the reaction, crude mixture was filtered with dichloromethane and concentrated under reduced pressure. The residue was purified by silica gel column chromatography eluting with 30% ethyl acetate in hexane to afford the desired product 12 (52 mg 70%) as an off-white solid. 1H NMR (400 MHz, CDCl3) δ 8.28 (dd, J=7.4, 1.0 Hz, 1H), 8.14 (dd, J=7.9, 0.9 Hz, 1H), 7.74 (s, 1H), 7.52 (t, J=7.7 Hz, 1H), 1.15 (d, J=0.7 Hz, 18H). 13C NMR (100 MHz, CDCl3) δ 171.6, 144.3, 141.9, 136.5 (d, J=16.1 Hz), 132.7 (d, J=2.9 Hz), 129.4, 128.4, 124.1, 123.3, 27.0, 20.2 (d, J=12.1 Hz). 19F NMR (376 MHz, CDCl3) δ−183.85. HRMS (APCI) m/z calcd for C17H22FO2SSi [M−H]+337.1099, found 337.1099.

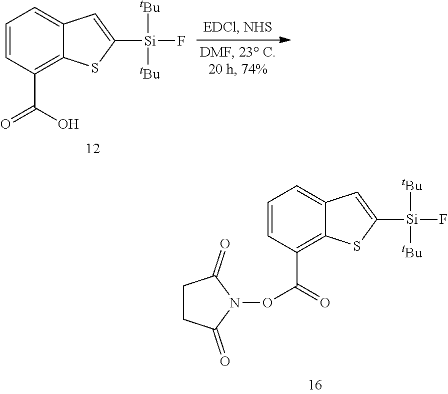 [SMILES: CC(C)(C)[Si](F)(c1cc2cccc(C(=O)O)c2s1)C(C)(C)C ; CC(C)(C)[Si](F)(c1cc2cccc(C(=O)ON3C(=O)CCC3=O)c2s1)C(C)(C)C]

### 2-(di-tert-butylfluorosilyl)benzo[b]thiophene-7-carboxylic acid N-succinimidyl ester (16)

To a stirred solution of carboxylic acid 12 (10 mg, 0.03 mmol) in DMF (0.12 mL) at 0° C., EDC.HCl (9 mg, 0.05 mmol) and N-hydroxysuccinimide (3.8 mg, 0.03 mmol) was added. The contents were stirred at room temperature for 20 h followed by extraction into dichloromethane. The organic phase was washed with brine, concentrated under reduce pressure and filtered through a short silica gel plug using 20% ethyl acetate in hexane to obtain NHS-ester 16 (9.5 mg 74%) as a white solid.

1H NMR (400 MHz, CDCl3) δ 8.33 (dd, J=7.5, 1.1 Hz, 1H), 8.19 (dd, J=7.9, 1.0 Hz, 1H), 7.74 (s, 1H), 7.53 (t, J=7.7 Hz, 1H), 1.11 (d, J=1.0 Hz, 1H). 13C NMR (100 MHz, CDCl3) δ 169.1, 161.5, 144.9, 141.9, 136.8, 132.9 (d, J=2.8 Hz), 130.5, 128.4, 124.2, 118.8, 26.9, 25.7, 20.2 (d, J=12.0 Hz). 19F NMR (376 MHz, CDCl3) δ −183.96. HRMS (APCI) m/z calcd for C21H26FNO4SSi [M+] 435.1330, found 435.1329.

### 2.5 Synthesis of Benzothiophene-Peptide Conjugates

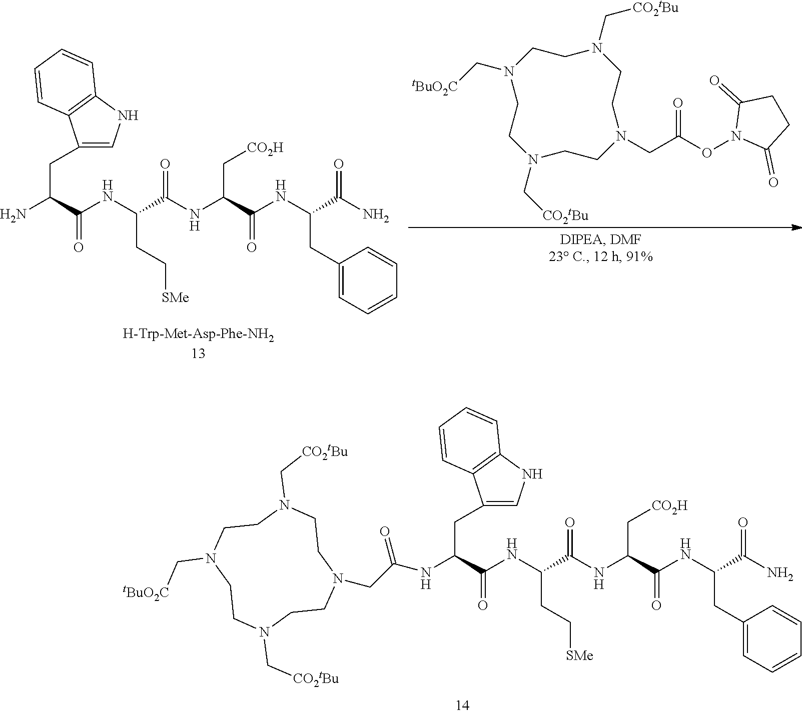 [SMILES: CSCC[C@H](NC(=O)[C@@H](N)Cc1c[nH]c2ccccc12)C(=O)N[C@@H](CC(=O)O)C(=O)N[C@@H](Cc1ccccc1)C(N)=O ; CC(C)(C)OC(=O)CN1CCN(CC(=O)ON2C(=O)CCC2=O)CCN(CC(=O)OC(C)(C)C)CCN(CC(=O)OC(C)(C)C)CC1 ; CSCC[C@H](NC(=O)[C@H](Cc1c[nH]c2ccccc12)NC(=O)CN1CCN(CC(=O)OC(C)(C)C)CCN(CC(=O)OC(C)(C)C)CCN(CC(=O)OC(C)(C)C)CC1)C(=O)N[C@@H](CC(=O)O)C(=O)N[C@@H](Cc1ccccc1)C(N)=O]

### tris(t-butyl)DOTA-Gastrin Conjugate (14)

To a stirred solution of gastrin tetrapeptide (H-Trp-Met-Asp-Phe-NH₂) 13 (7.31 mg, 12.25 μmol) in DMF (0.15 mL) was added DOTA-mono-NHS tris(t-Bu ester) (10 mg, 12.25 μmol) and DIPEA (4.2 μL, 24.50 μmol) at 0° C. Reaction mixture was stirred at room temperature for 12 h. The crude product was purified by semi-preparative reverse phase HPLC (10% to 90% CH₃CN in water (both with 0.1% TFA) over 30 minutes, 4 mL/min flow rate; UV 220 nm). The product fractions were lyophilized to obtain desired peptide conjugate 14 (12.8 mg, 91%) as a white solid. The identity of the conjugate was confirmed by HRMS. HRMS (ESI) m/z calcd for C57H87N10O13S [M+H]+ 1151.6175, found 1151.6178.

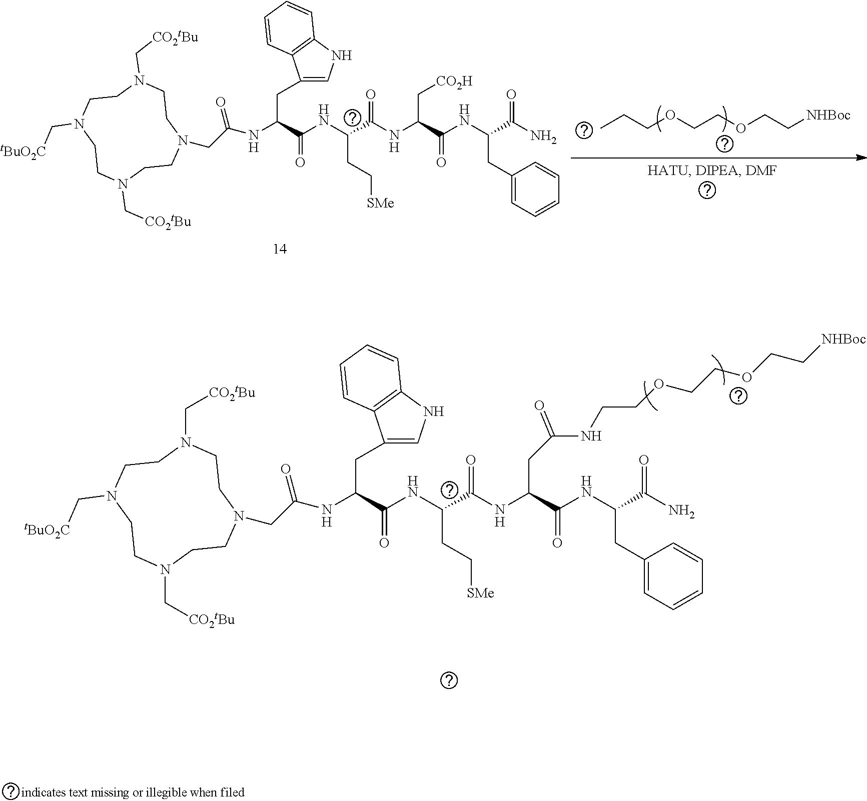

### N-boc-PEGylated tris(t-butyl)DOTA-Gastrin conjugate (15-Boc)

To a solution of peptide conjugate 14 (14 mg, 12.16 μmol) in DMF (0.2 mL) cooled to 0-4° C. (in ice bath), was added HATU (5.54 mg, 14.59 μmol). The contents were stirred at 0-4° C. for 5 min. To the reaction mixture, was added tert-butyl (2-(2-(2-(2-aminoethoxy) ethoxy)ethoxy)ethyl)carbamate (7.12 mg, 14.59 μmol) and DIPEA (6.3 μL, 36.47 μmol) and the reaction was stirred at room temperature for 18 hrs. The crude reaction was purified by semi-preparative HPLC (10% to 90% CH3CN in water (both with 0.1% TFA) over 30 minutes, 4 mL/min flow rate; UV 220 nm). The product fractions were lyophilized to get PEGylated peptide conjugate 15-Boc (15.7 mg, 90%) as a white solid. The identity of the conjugate was confirmed by HRMS. HRMS (ESI) m/z calcd for C70H112N12O17SNa [M+Na]+1447.7886 found 1447.7916, and [M/2+H]2+ calcd 713.4067, found 713.4096.

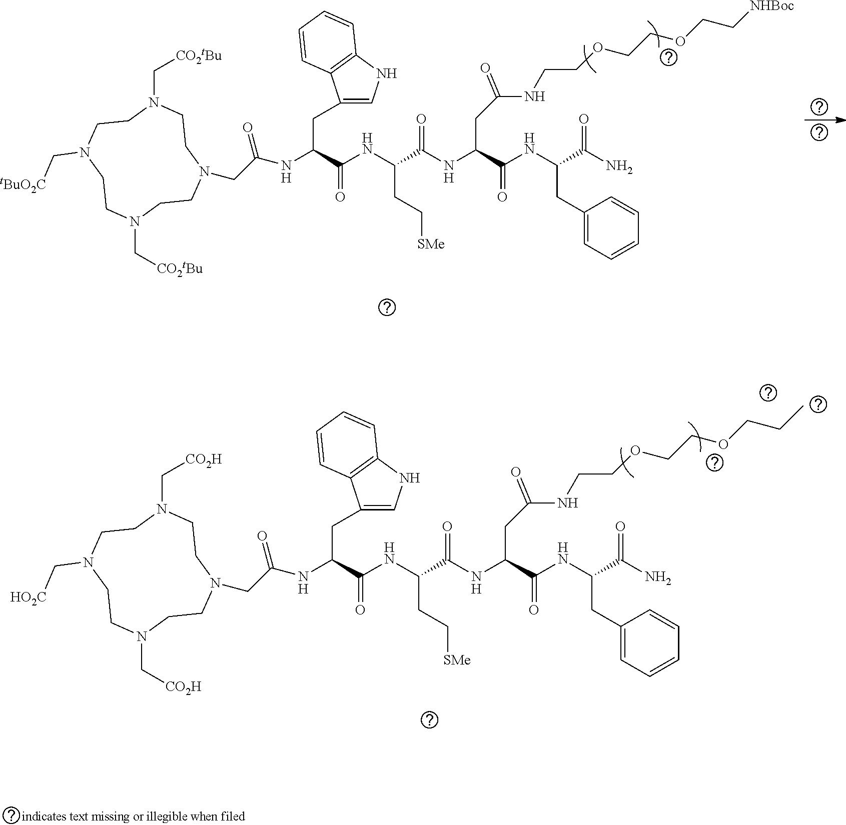

### PEGylated DOTA-Gastrin Conjugate (15)

Boc protected peptide conjugate 15-Boc (15.4 mg) was taken in dichloromethane (0.2 mL) and trifluoroacetic acid (0.5 mL) and stirred at room temperature for 8 h. The solvent was evaporated under reduced pressure and crude residue was used in the following reaction without any purification. HRMS (ESI) m/z calcd for C53H81N12O15S [M+H]+ 1157.5665, found 1157.5687.

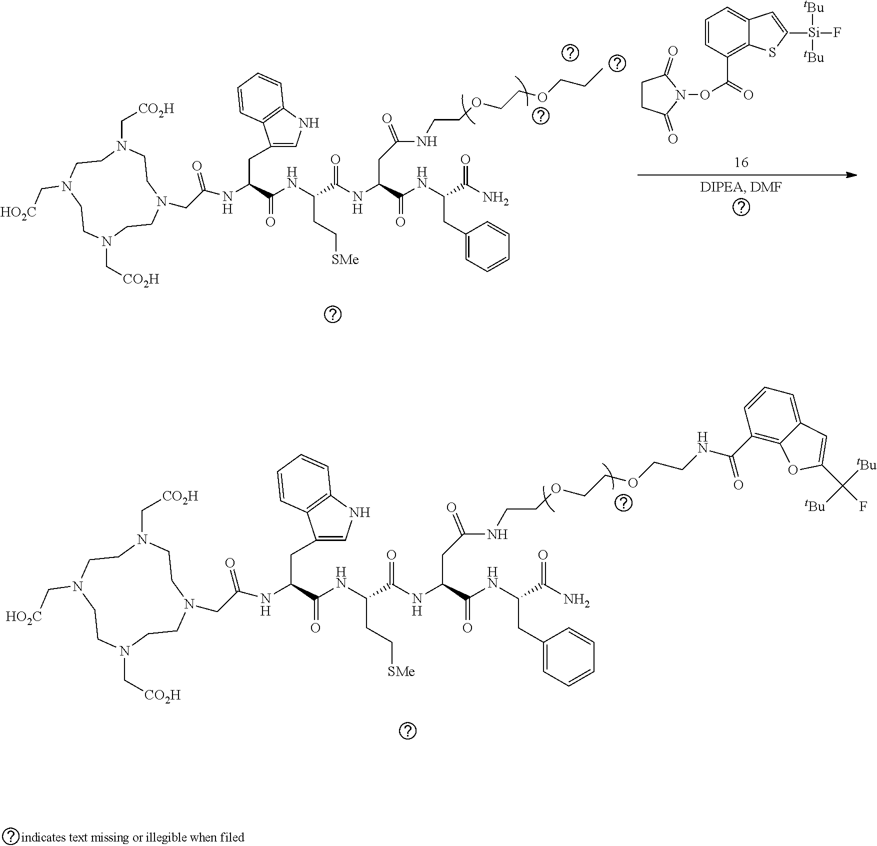

### Benzothiophene-SiFA-Peptide Conjugate (17)

To a stirred solution of PEGylated DOTAGastrin conjugate 15 (6 mg, 4.76 μmol) in 150 μL DMF and 2.5 μL DIPEA was added Nhydroxysuccinimidyl ester 16 (2 mg, 4.53 μmol) at 0° C. The crude reaction was stirred at room temperature for 20 h. The crude residue was purified by semi-preparative HPLC (10% to 90% CH₃CN in water (both with 0.1% TFA) over 30 minutes, 4 mL/min flow rate; UV 220 nm). The product fractions were lyophilized to afford benzothiophene-SiFA-peptide conjugate 17 (3.7 mg, 54%) and recovered NHS-ester 16. The identity of the conjugates were confirmed by HRMS. HRMS (ESI) caled for C70H101FN12O16S2Si [M−H]− 1475.6580 found 1475.6617 and m/z calcd for C70H101FN12O16S2Si [M/2+H]2+739.3399, found 739.3410.

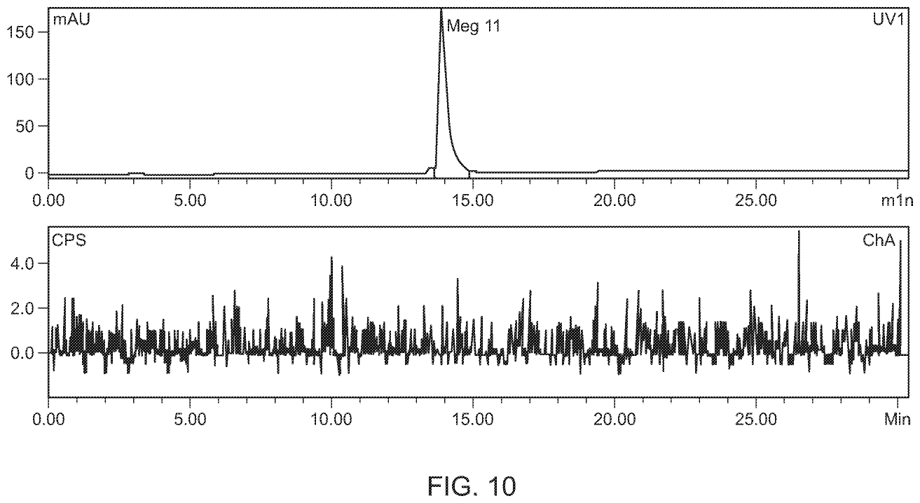

FIG. 10 shows data from an analytical HPLC chromatograph of purified benzothiophene-SiFA-peptide conjugate 17.

### 3. Radiochemistry

### 3.1 General Materials and Methods

No-carrier-added [¹⁸F]fluoride was produced by the 18O(p,n)¹⁸F nuclear reaction in a Siemens RDS-112 cyclotron at 11 MeV using a 1 mL tantalum target with havar foil. The solvents and reagents were commercially available and used without further purification. HPLC grade acetonitrile and trifluoroacetic acid were purchased from Fisher Scientific. Anhydrous acetonitrile, dimethyl sulfoxide and tetraethylammonium bicarbonate were purchased from Sigma-Aldrich. Sterile product vials were purchased from Hollister-Stier. QMA-light Sep-Paks and tC18 light cartridges were purchased from Waters Corporation. Radio-TLCs were analyzed using a miniGITA*TLC scanner. HPLC purifications were performed on a Knauer Smartline HPLC system with inline Knauer UV (254 nm) detector and gamma-radiation coincidence detector and counter (Bioscan Inc.). Semi-preprative HPLC was performed using Phenomenex reverse-phase Luna column (10×250 mm, 5 μm) with a flow rate of 4 mL/min. Final purity and identity of compounds were determined by analytical HPLC analysis performed with a Phenomenex reverse phase Luna column (4.6×250 mm, 5 μm) with a flow rate of 1.2 mL/min or 1 mL/min. All chromatograms were collected by a GinaStar (Raytest) analog to digital converter and GinaStar software.

### Preparation of [¹⁸F]tetraethylammonium fluoride ([¹⁸F]TEAF)

Dry [¹⁸F]TEAF was prepared using an ELIXYS automated radiosynthesizer (Sofie Biosciences). [¹⁸F]Fluoride was delivered to the ELIXYS in [18O]H2O (1 mL) via nitrogen gas push and trapped on a QMA cartridge to remove the [18O]H2O. Trapped [¹⁸F]fluoride was subsequently eluted into the reaction vial using a solution containing Et4NHCO3 (1.8-2.0 mg, ˜10 μmol) in acetonitrile and water (1 mL, 8:2) (QMA cartridge was flipped before elution step). Contents in the reaction vial were evaporated by heating the vial to 110° C. while applying a vacuum for 3.5 min, with stirring. Acetonitrile (1.3 mL) was passed through the QMA cartridge to wash remaining activity into the reaction vial. The combined contents in the reaction vial were dried by azeotropic distillation (heating to 110° C. under vacuum) for 2 min. Anhydrous acetonitrile (1.3 mL) was directly added to the reaction vial and azeotropic distillation was repeated once more until dryness, approximately 3-4 min. The reaction vial was cooled to 30° C. under nitrogen pressure and acetonitrile (1 mL) was added to provide anhydrous [¹⁸F]TEAF which was used for subsequent reactions. *Note: In some cases, an alternate protocol using methanol as the eluent was employed to obtain dry [¹⁸F]TEAF. Briefly, the QMA cartridge washed with 1 mL methanol followed by 5 mL air. [¹⁸F]Fluoride was trapped on the QMA and eluted with a solution of tetraethylammonium bicarbonate (1.5-2 mg) in methanol (0.8 mL, cartridge was flipped while elution). Additional methanol (0.8 mL) was eluted through the QMA and the methanol was evaporated at 70° C., under vacuum to obtain dry [¹⁸F]TEAF.

### 3.2 Isotopic Exchange Reactions and Characterization of 1⁸F-Labeled Compounds

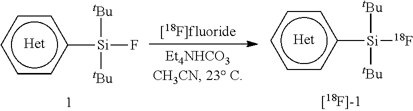

### General Experimental Procedure

Isotopic exchange reactions were conducted in 1 mL Eppendorf tube containing hetroarylfluorosilanes 1 in dry acetonitrile (3 mM stock solution, 50 μL). To the Eppendorf tube, was added 0.5-1 mCi of [¹⁸F]TEAF in 100-150 μL of dry acetonitrile and the contents were left at room temperature for 2 min without stirring. An aliquot of the crude reaction mixture was spotted on a silica gel coated TLC plate, developed in a glass chamber using [¹⁸F]fluoride Het Si tBu tBu F Het Si tBu tBu ¹⁸F Et4NHCO3 CH3CN, 23° C. 1 [¹⁸F]-1 S15 acetonitrile (100%) or acetonitrile:water (95:5) as the eluent and analyzed by radio-TLC using a miniGITA*TLC scanner. The radiochemical conversion (RCC) was calculated by dividing the integrated area of the ¹⁸F-fluorinated product peak by the total integrated area of all peaks on the TLC and multiplying by 100 to convert to percentage units. Isotopic exchange and purity was confirmed by analytical HPLC by co-injecting with the 19F-reference standard (UV absorbance at 254 nm). An aliquot of the crude reaction mixture (10 μL) was added to the 19F-reference standard (1 mg/mL) in acetonitrile (10 μL) and the sample was injected into the analytical HPLC.

### 3.3 Optimization Screening

High Base Vs Low Base.

[¹⁸F]TEAF was obtained by eluting fluoride ion with high base or low base, following the protocol described above. Elution with high base led to the formation of hydrolyzed products as seen in analytical HPLC (UV impurities) and the radiochemical conversions dropped over time.

TABLE S1
Reactions were conducted with HetSiFA 6 using 50 μmol Et4NHCO3
High Base

| Concentration | | | | | |
|---|---|---|---|---|---|
| nmol (run) | 2 min | 5 min | 15 min | 30 min | 60 min |
| 150(1) | 93.45 | 88.15 | 83.25 | 78.24 | 74.23 |
| 150(2) | 94.67 | 88.12 | 82.24 | 76.14 | 70.12 |
| 150(3) | 96.46 | 89.14 | 81.47 | 75.49 | 71.35 |
| Average | 94.86 | 88.47 | 82.32 | 76.62 | 71.9 |
| Standard Dev. | 1.51 | 0.58 | 0.89 | 1.43 | 2.11 |
| 100(1) | 95.73 | 90.12 | 81.12 | 75.23 | 70.25 |
| 100(2) | 94.23 | 90.26 | 79.53 | 73.15 | 69.42 |
| 100(3) | 94.38 | 89.92 | 78.47 | 75.24 | 68.28 |
| Average | 94.78 | 90.1 | 79.70 | 74.54 | 69.32 |
| Standard Dev. | 0.83 | 0.17 | 1.33 | 1.20 | 0.98 |
| 50(1) | 88.15 | 85.21 | 78.21 | 72.12 | 68.36 |
| 50(2) | 88.2 | 84.56 | 77.63 | 71.25 | 66.72 |
| 50(3) | 85.21 | 81.03 | 76.41 | 70.52 | 65.23 |
| Average | 87.18 | 83.60 | 77.41 | 71.29 | 66.77 |
| Standard Dev. | 1.71 | 2.25 | 0.92 | 0.80 | 1.57 |

TABLE S2
Reactions were conducted with HetSiFA 6 using 5.75 μmol Et4NHCO3
Low Base

| Concentration | | | |
|---|---|---|---|
| nmol (run) | 2 min | 15 min | 30 min |
| 150(1) | 92.84 | 94.11 | 95.39 |
| 150(2) | 94.33 | 96.11 | 95.66 |
| 150(3) | 92.21 | 92.05 | 97.45 |
| 150(4) | 94.67 | — | — |
| Average | 93.51 | 94.09 | 96.16 |
| Standard Dev. | 1.18 | 2.03 | 1.11 |
| 100(1) | 92.13 | 91.21 | 91.82 |
| 100(2) | 92.77 | 93.1 | 93.39 |
| 100(3) | 92.12 | 93.85 | 93.69 |
| 100(4) | 89.92 | 90.85 | 92.53 |
| Average | 91.74 | 92.25 | 92.85 |
| Standard Dev. | 1.25 | 1.45 | 0.84 |
| 50(1) | 88.97 | 93.44 | 95.43 |
| 50(2) | 89.52 | 94.48 | 93.83 |
| 50(3) | 90.83 | 93.85 | 93.03 |
| Average | 89.77 | 93.92 | 94.09 |
| Standard Dev. | 0.95 | 0.523 | 1.22 |
| 10(1) | 83.52 | 87.52 | 93.51 |
| 10(2) | 82.54 | 85.86 | 92.26 |
| 10(3) | 85.52 | 88.87 | 90.94 |
| Average | 83.86 | 87.42 | 92.23 |
| Standard Dev. | 1.52 | 1.50 | 1.29 |

TABLE S3
Reactions were conducted with HetSiFA 12.

| | | Elution | Time | |
|---|---|---|---|---|
| Et₄NHCO₃ | Elution solvert | efficiency (%) | (min)^{b} | RCC (%) |
| 9.0 (47.1) | Acetonitrile/water | 96 | 2 | 93 |
| | | | 15 | 85 |
| | | | 30 | 65 |
| 5.0 (26.2) | Acetonitrile/water | 78 | 2 | 90 |
| | | | 15 | 80 |
| | | | 30 | 75 |
| 40.0 (20.9) | Methanol | 50^{a} | 2 | 88 |
| | | | 15 | 82 |
| | | | 30 | 76 |
| 2.0 (10.5) | Methanol | 35^{a} | 2 | 92 |
| | | | 15 | 90 |
| | | | 30 | 90 |
| 2.0 (10.5) | Acetonitrile/water | 64^{a} | 2 | 95 |
| | | | 15 | 97 |
| | | | 30 | 97 |
| | | | 60 | 95 |
| 1.5 (7.8) | Acetonitrile/water | 60^{a} | 2 | 91 |
| 1.1 (5.8 | Acetonitrile/water | 55^{a} | 2 | 88 |
| 1.2 | | | | |

^{a}QMA cartridge was flipped to elute in the opposite direction as [¹⁸F]fluoride was trapped.
^{b}Reactions were conducted with 150 nmol HetSiFA 12 and left at room temperature without stirring.

TABLE S4
Stability screen of HetSiFA [¹⁸F]-12 in acetonitrile
with 1.8 mg (9.4 μmol) Et4NHCO3 used for elution.

| Time (min) | RCC (%)^{a} |
|---|---|
| 2 | 95 |
| 5 | 96 |
| 15 | 97 |
| 30 | 97 |
| 60 | 97 |

^{a}Reaction was conducted at room temperature.

TABLE S5
Effect of precursor concentration of HetSiFA 12 on RCC.

| Precursor amount (nmol) | RCC(%)^{a} |
|---|---|
| 5 | 58 |
| 25 | 83 |
| 50 | 86 |
| 100 | 94 |
| 150 | 96 |

^{a}Reaction conditions: Et4NHCO3 (1.8 mg, 9.4 μmol) in 150 μL acetonitrile. RCCs were obtained after 2 min standing at room temperature.

### 3.4 Radio-TLC/Radio-HPLC Analysis of 1⁸F-Labeled HetSiFAs

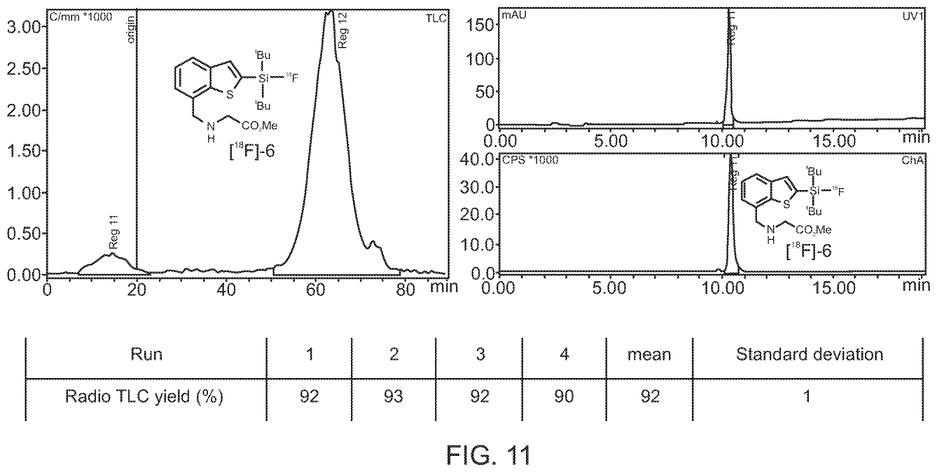

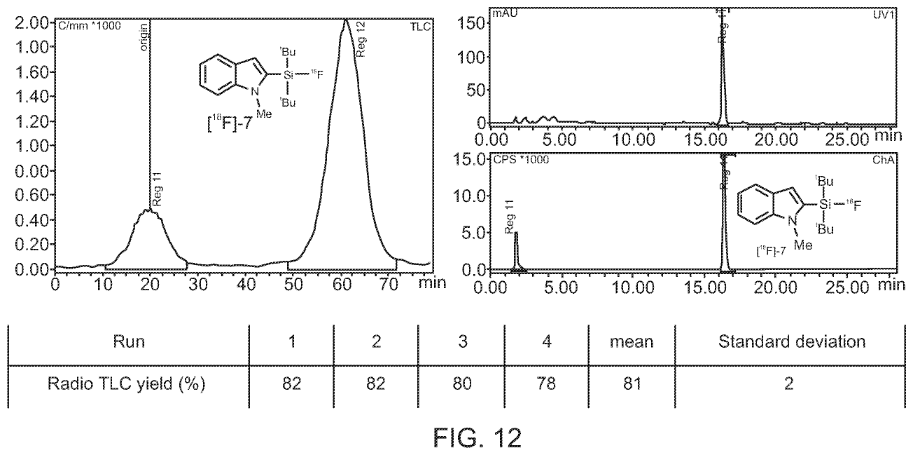

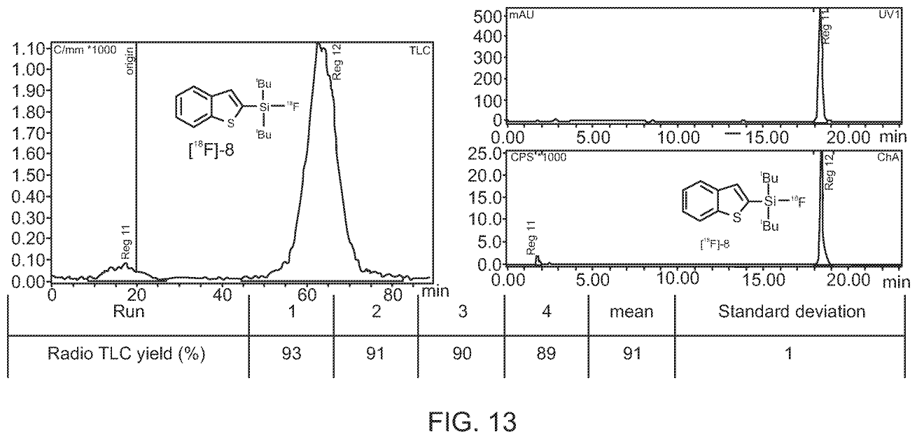

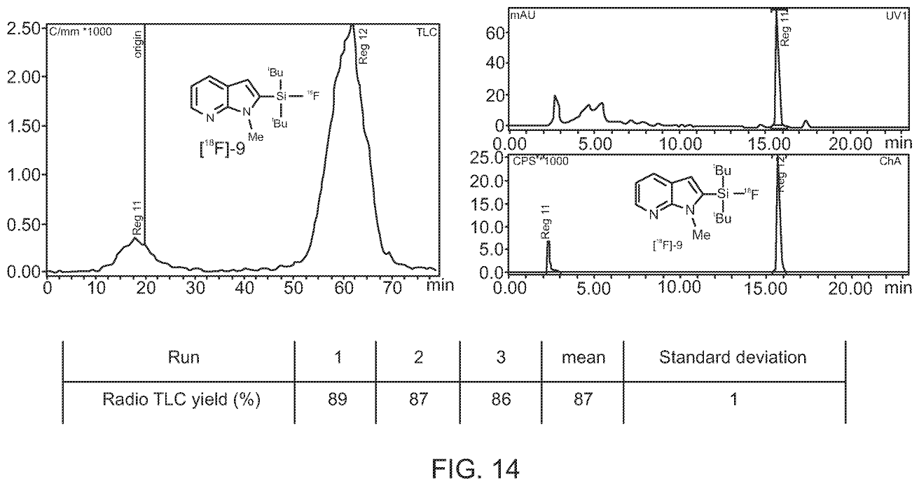

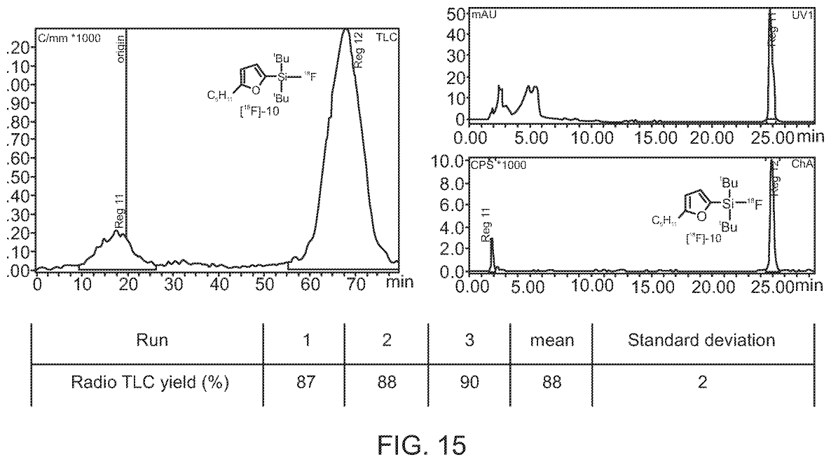

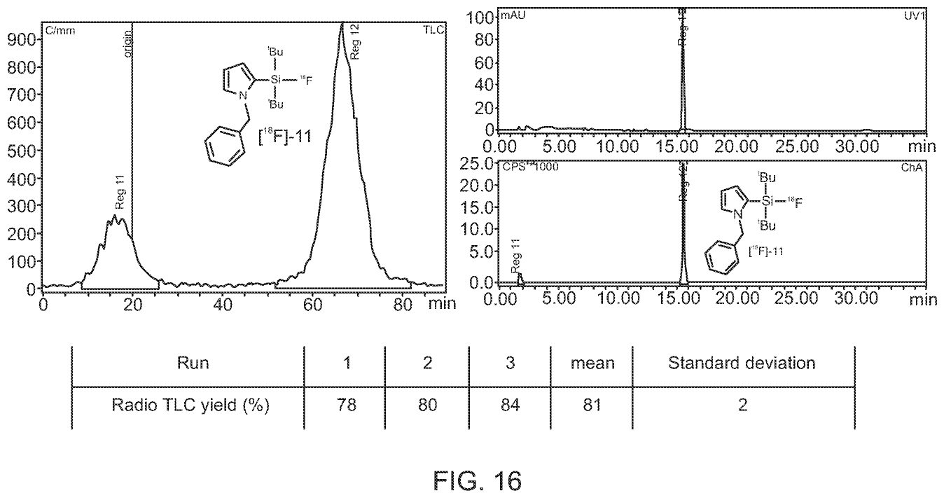

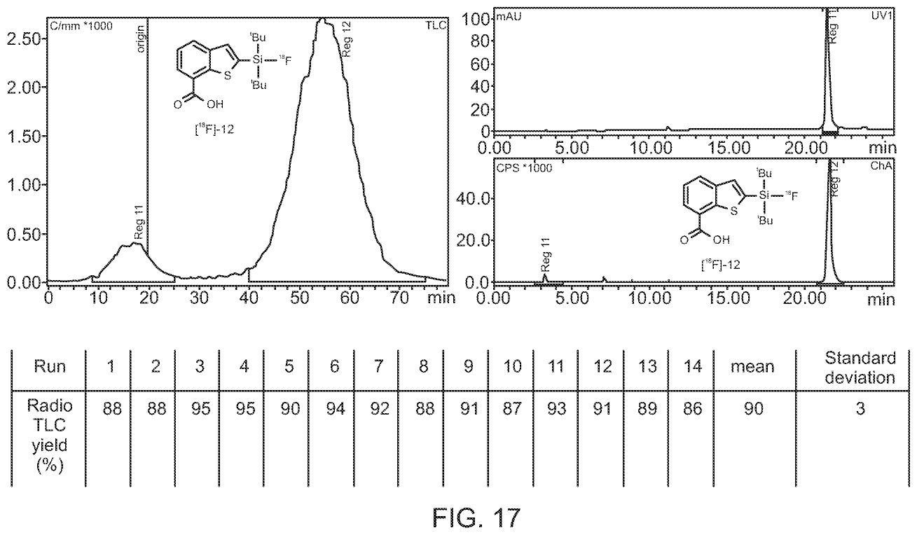

FIGS. 11-17 shows data from an isotopic exchange and characterizations of various embodiments of the invention.

### 3.5 Automated Radiolabeling of Benzothiophene-SiFA-Peptide Conjugate 17

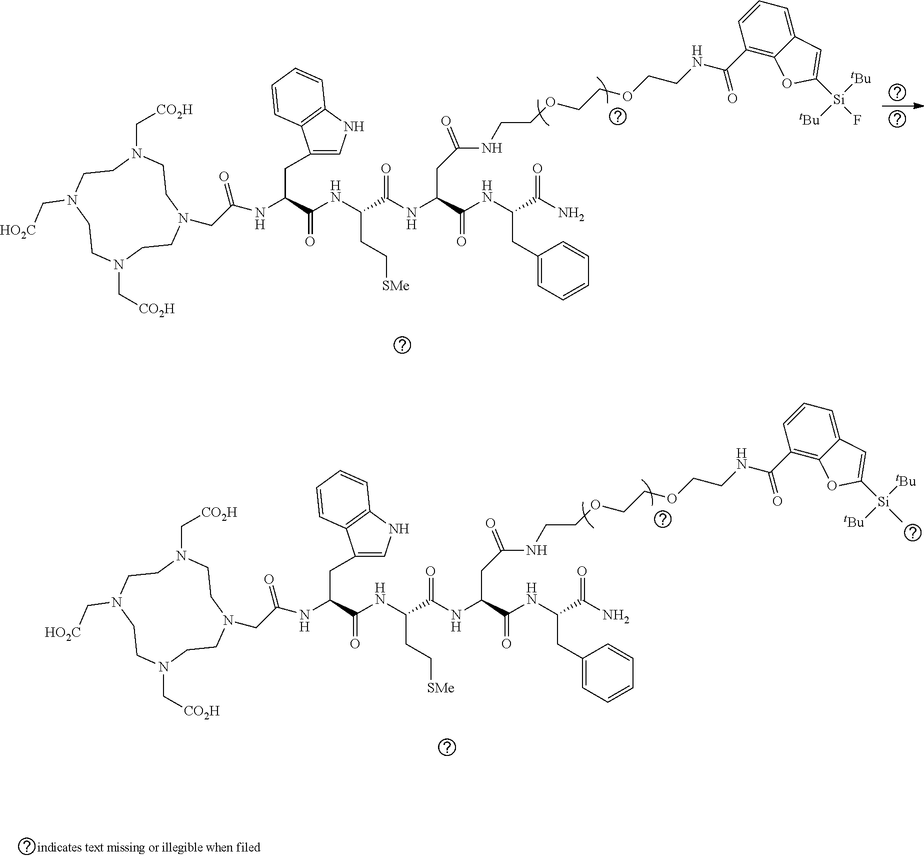

Using an ELIXYS radiosynthesis module, benzothiophene-SiFA-Peptide conjugate 17 in acetonitrile (1 mM solution, 250 μL) was added to anhydrous [¹⁸F]TEAF (˜12 mCi) and contents were kept at 23° C. for 2 min without stirring. The crude mixture was diluted with 10 mL 0.01 M HEPES (pH=4) and passed through a C18 cartridge to afford [¹⁸F]-17. Residual solvent and [¹⁸F]fluoride were removed by flushing the C18 cartridge with water (˜2 mL). [¹⁸F]-17 was eluted with ethanol (250-500 μL) and diluted with saline to a final formulation of 5-8% ethanol in saline.

TABLE S6
Isotopic exchange of peptide conjugate 17 to afford [¹⁸F]-17.

| | Initial | Final Activity | | % RCY^{c} |
|---|---|---|---|---|
| | [¹⁸F]TEAF for | after | | (isolated, |
| | IEX reaction | formulation | | non-decay |
| Et₄NHCO₃ | (mCi) | (mCi) | % RCC^{b} | corrected) |
| 1.8 (9.4) | 11.5 | 7.5 | 95 | 65 |
| 1.8 (9.4) | 12.5 | 6.6 | 93 | 53 |
| 1.5 (7.8) | 11.2 | 6.2 | 95 | 55 |

| | |
|---|---|
| 94 ± 1 | 58 ± 6 |

^{a}Standard elution with acetonitrile:water as described above.
^{b}RCC determined by radio-TLC, before formulation.
^{c}Isolated RCY calculated after formulation; formulation was not optimized.

### 3.6 Molar Activity of Benzothiophene-SiFA-Peptide Conjugate [¹⁸F]-17

A calibration curve was generated from standard solutions of 17, by measuring the UV absorbance at different concentrations. The activity of [¹⁸F]-17 injected divided by the concentration of the product measured from the calibration curve afforded the molar activity. The molar activity of [¹⁸F]-17 was calculated to be 0.032±0.015 Ci/μmol.

### 4. MicroPET/CT In Vivo Imaging Experiments

### 4.1 Methods

Animal studies were approved by the UCLA Animal Research Committee and carried out according to the guidelines of the Department of Laboratory Animal Medicine at UCLA. Female C57BL6 mice were injected intravenously via tail vein with approximately 2.2 MBq (60 μCi) of [¹⁸F]-17. Animals were kept warm on heating pads throughout the imaging procedures. At 1 and 2 h after tracer injection, mice were anesthetized with 2% isoflurane in oxygen and placed in dedicated Genisys8 imaging chambers for PET/CT imaging on the Genisys8 PET/CT (Sofie Biosciences). PET scans were acquired for 10 min with an energy window of 150-650 keV reconstructed using ML-EM, followed by CT acquisition. All PET images were corrected for CTbased photon attenuation, detector normalization and radioisotope decay (scatter correction was not applied) and converted to units of percent injected dose per gram (% ID/g). Images were analyzed by drawing regions-of-interest (ROI) in select tissues using AMIDE v1.0.5. See e.g. Loening et al., Mol. Imaging 2003, 3, 131.

TABLE S7
Biodistribution of [¹⁸F]-17 in C57BL6 mice following
PET quantification (% ID/g), 1 and 2 h post-injection.

| Organ | 1 h | 2 h |
|---|---|---|
| GI | 107 ± 24 | 122 ± 48 |
| Bladder | 35 ± 12 | 30 ± 7 |
| Gallbladder | 33 ± 14 | 28 ± 9 |
| Bone (knee) | 8.0 ± 0.65 | 8.2 ± 0.56 |
| Liver | 5.0 ± 0.07 | 2.4 ± 0.47 |
| Kidney | 2.9 ± 0.62 | 2.2 ± 0.38 |
| Heart | 2.0 ± 0.25 | 1.2 ± 0.26 |
| Brain | 0.44 ± 0.06 | 0.33 ± 0.03 |
| Muscle | 0.36 ± 0.06 | 0.27 ± 0.11 |

### REFERENCES

(1) Ting, R.; Adam, M. J.; Ruth, T. J.; Perrin, D. M. J. Am. Chem. Soc. 2005, 127, 13094-13095.
(2) Mu, L.; Höhne, A.; Schubiger, P. A.; Ametamey, S. M.; Graham, K.; Cyr, J. E.; Dinkelborg, L.; Stellfeld, T.; Srinivasan, A.; Voigtmann, U.; Klar, U. Angew. Chem., Int. Ed. 2008, 47, 4922-4925.
(3) Mu, L.; August Schubiger, P.; Ametamey, S. M. Curr. Radiopharm. 2010, 3, 224-242.
(4) Wa\ddot{n} gler, C.; Kostikov, A.; Zhu, J.; Chin, J.; Wa\ddot{n} gler, B.; Schirrmacher, R. Appl. Sci. 2012, 2, 277-302.
(5) Wängler, C.; Niedermoser, S.; Chin, J.; Orchowski, K.; Schirrmacher, E.; Jurkschat, K.; Iovkova-Berends, L.; Kostikov, A. P.; Schirrmacher, R.; Wa\ddot{n} gler, B. Nat. Protoc. 2012, 7, 1946.
(6) Bernard-Gauthier, V.; Wa\ddot{n} gler, C.; Schirrmacher, E.; Kostikov, A.; Jurkschat, K.; Wa\ddot{n} gler, B.; Schirrmacher, R. BioMed Res. Int. 2014, 2014, 1-20.
(7) Ting, R.; Harwig, C.; auf dem Keller, U.; McCormick, S.; Austin, P.; Overall, C. M.; Adam, M. J.; Ruth, T. J.; Perrin, D. M. J. Am. Chem. Soc. 2008, 130, 12045-12055.
(8) Burke, B. P.; Clemente, G. S.; Archibald, S. J. Contrast Media Mol. Imaging 2015, 10, 96-110.
(9) Chansaenpak, K.; Vabre, B.; Gabbaï, F. P. Chem. Soc. Rev. 2016, 45, 954-971.
(10) McBride, W. J.; D'Souza, C. A.; Sharkey, R. M.; Karacay, H.; Rossi, E. A.; Chang, C.-H.; Goldenberg, D. M. Bioconjugate Chem. 2010, 21, 1331-1340.
(11) D'Souza, C. A.; McBride, W. J.; Sharkey, R. M.; Todaro, L. J.; Goldenberg, D. M. Bioconjugate Chem. 2011, 22, 1793-1803.
(12) Vabre, B.; Chansaenpak, K.; Wang, M.; Wang, H.; Li, Z.; Gabbaï, F. P. Chem. Commun. 2017, 53, 8657-8659.
(13) Hong, H.; Zhang, L.; Xie, F.; Zhuang, R.; Jiang, D.; Liu, H.; Li, J.; Yang, H.; Zhang, X.; Nie, L.; Li, Z. Nat. Commun. 2019, 10, 989.
(14) Bernard-Gauthier, V.; Lepage, M. L.; Waengler, B.; Bailey, J. J.; Liang, S. H.; Perrin, D. M.; Vasdev, N.; Schirrmacher, R. J. Nucl. Med. 2018, 59, 568-572.
(15) Bemard-Gauthier, V.; Bailey, J. J.; Liu, Z.; Wa\ddot{n} gler, B.; Wängler, C.; Jurkschat, K.; Perrin, D. M.; Schirrmacher, R. Bioconjugate Chem. 2016, 27, 267-279.
(16) Schirrmacher, R.; Bradtmöller, G.; Schirrmacher, E.; Thews, O.; Tillmanns, J.; Siessmeier, T.; Buchholz, H. G.; Bartenstein, P.; Wängler, B.; Niemeyer, C. M.; Jurkschat, K. Angew. Chem., Int. Ed. 2006, 45, 6047-6050.
(17) Rosenthal, M. S.; Bosch, A. L.; Nickles, R. J.; Gatley, S. J. Int. J. Appl. Radiat. Isot. 1985, 36.
(18) Liu, Z.; Li, Y.; Lozada, J.; Pan, J.; Lin, K.-S.; Schaffer, P.; Perrin, D. M. J. Labelled Compd. Radiopharm. 2012, 55, 491-496.
(19) Liu, Z.; Hundal-Jabal, N.; Wong, M.; Yapp, D.; Lin, K. S.; Bénard, F.; Perrin, D. M. MedChemComm 2014, 5, 171-179.
(20) Niedermoser, S.; Chin, J.; Wa\ddot{n} gler, C.; Kostikov, A.; Bernard-Gauthier, V.; Vogler, N.; Soucy, J.-P.; McEwan, A. J.; Schirrmacher, R.; Wängler, B. J. Nucl. Med. 2015, 56, 1100-1105.
(21) Liu, Z.; Pourghiasian, M.; Radtke, M. A.; Lau, J.; Pan, J.; Dias, G. M.; Yapp, D.; Lin, K.-S.; Bénard, F.; Perrin, D. M. Angew. Chem., Int. Ed. 2014, 53, 11876-11880.
(22) Liu, Z.; Pourghiasian, M.; Beń ard, F.; Pan, J.; Lin, K.-S.; Perrin, D. M. J. Nucl. Med. 2014, 55, 1499-1505.
(23) Liu, Z.; Li, Y.; Lozada, J.; Schaffer, P.; Adam, M. J.; Ruth, T. J.; Perrin, D. M. Angew. Chem., Int. Ed. 2013, 52, 2303-2307.
(24) Schirrmacher, E.; Wa\ddot{n} gler, B.; Cypryk, M.; Bradtmöller, G.; Schäfer, M.; Eisenhut, M.; Jurkschat, K.; Schirrmacher, R. Bioconjugate Chem. 2007, 18, 2085-2089.
(25) Litau, S.; Niedermoser, S.; Vogler, N.; Roscher, M.; Schirrmacher, R.; Fricker, G.; Wängler, B.; Wängler, C. Bioconjugate Chem. 2015, 26, 2350-2359.
(26) Toutov, A. A.; Liu, W.-B.; Betz, K. N.; Fedorov, A.; Stoltz, B. M.; Grubbs, R. H. Nature 2015, 518, 80.
(27) Hansen, M. M.; Clayton, M. T.; Godfrey, A. G.; Grutsch, J. L., Jr.; Keast, S. S.; Kohlman, D. T.; McSpadden, A. R.; Pedersen, S. W.; Ward, J. A.; Xu, Y.-C. Synlett 2004, 8, 1351-1354.
(28) Baranski, A.-C.; Scha\ddot{f} er, M.; Bauder-Wüst, U.; Wacker, A.; Schmidt, J.; Liolios, C.; Mier, W.; Haberkorn, U.; Eisenhut, M.; Kopka, K.; Eder, M. Bioconjugate Chem. 2017, 28, 2485-2492.
(29) Eder, M.; Löhr, T.; Bauder-Wüst, U.; Reber, M.; Mier, W.; Schäfer, M.; Haberkorn, U.; Eisenhut, M. J. Nucl. Med. 2013, 54, 1327-1330.
(30) Richter, S.; Wuest, M.; Bergman, C. N.; Way, J. D.; Krieger, S.; Rogers, B. E.; Wuest, F. Bioconjugate Chem. 2015, 26, 201-212.
(31) Roxin, Á.; Zhang, C.; Huh, S.; Lepage, M.; Zhang, Z.; Lin, K.-S.; Bénard, F.; Perrin, D. M. Bioconjugate Chem. 2019, 30, 1210-1219.
(32) Lepage, M. L.; Kuo, H.-T.; Roxin, Á.; Huh, S.; Zhang, Z.; Kandasamy, R.; Merkens, H.; Kumlin, J. O.; Limoges, A.; Zeisler, S. K.; Lin, K-S.; Bénard, F.; Perrin, D. M. ChemBioChem 2019, DOI: 10.1002/cbic.201900632.
(33) Hong, H.; Zhang, L.; Xie, F.; Zhuang, R.; Jiang, D.; Liu, H.; Li, J.; Yang, H.; Zhang, X.; Nie, L.; Li, Z. Nat. Commun. 2019, 10, 989.
(34) Sergeev, M.; Lazari, M.; Morgia, F.; Collins, J.; Javed, M. R.; Sergeeva, O.; Jones, J.; Phelps, M. E.; Lee, J. T.; Keng, P. Y.; van Dam, R. M. Commun. Chem. 2018, 1, 10.
(35) Li, Y.; Liu, Z.; Harwig, C. W.; Pourghiasian, M.; Lau, J.; Lin, K-S.; Schaffer, P.; Benard, F.; Perrin, D. M. Am. J. Nucl. Med. Mol. Imaging 2013, 3, 57-70.
(36) Ilhan, H.; Todica, A.; Lindner, S.; Boening, G.; Gosewisch, A.; Wa\ddot{n}gler, C.; Wa\ddot{n}gler, B.; Schirrmacher, R.; Bartenstein, P. Eur. J. Nucl. Med. Mol. Imaging 2019, 46, 2400-2401.
(37) Wurzer, A.; Di Carlo, D.; Schmidt, A.; Beck, R.; Eiber, M.; Schwaiger, M.; Wes ter, H. J. Nuc 1. Med. 2019, 60, jnumed.119.234922.

TABLE 1
Optimization of isotope exchange of HetSiFA ™ 6
to form [¹⁸F]−6^{a}

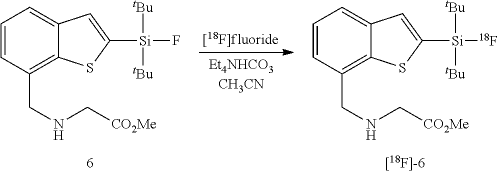 [SMILES: COC(=O)CNCc1cccc2cc([Si](F)(C(C)(C)C)C(C)(C)C)sc12 ; COC(=O)CNCc1cccc2cc([Si]([18F])(C(C)(C)C)C(C)(C)C)sc12]

| Entry | 6 (nmol) | time (min) | RCC (%)^{b} | RCC (%)^{c} |
|---|---|---|---|---|
| 1 | 150 | 2 | 95 ± 2 | 94 ± 1 |
| 2 | 150 | 5 | 88 ± 1 | — |
| 3 | 150 | 15 | 82 ± 1 | 94 ± 2 |
| 4 | 150 | 30 | 77 ± 1 | 96 ± 1 |
| 5 | 100 | 2 | 95 ± 1 | 92 ± 1 |
| 6 | 100 | 5 | 90 ± 0 | — |
| 7 | 100 | 15 | 79 ± 1 | 92 ± 1 |
| 8 | 100 | 30 | 74 ± 1 | 93 ± 1 |
| 9 | 50 | 2 | 87 ± 2 | 89 ± 1 |
| 10 | 50 | 5 | 84 ± 2 | — |
| 11 | 50 | 15 | 77 ± 1 | 93 ± 1 |
| 12 | 50 | 30 | 71 ± 1 | 94 ± 1 |
| 13 | 10 | 2 | — | 83 ± 2 |
| 14 | 10 | 15 | — | 87 ± 2 |
| 15 | 10 | 30 | — | 92 ± 1 |

^{a}Conditions: precursor, [¹⁸F]fluoride (400-700 μCl), CH₃CN (150 μL), 23° C. [¹⁸F]fluoride eluted with CH₃CH:H₂O (0.8 mL:0.2 mL). RCC was determined by radio-TLC. Reactions performed in triplicate.
^{b}Et₄NHCO₃ (50 μmol) was used to elute [¹⁸F]fluoride.
^{c}Et₄NHCO₃ (5.75 μmol) was used to elute [¹⁸F]fluoride.

1. A composition of matter comprising:
a compound comprising an aromatic heterocyclic core comprising a mono- or polycyclic-aromatic chemical moiety featuring one or more heteroatoms, and
at least one functionality attached to the aromatic heterocyclic core, wherein the functionality comprises:
a chemical moiety Q comprising Si;
wherein Q is attached to the aromatic heterocyclic core via a covalent chemical bond;
an integer number m of atoms or functional groups X associated with Q,
wherein m≥0, and each X, if any, is independently chosen such that it can be displaced by a nucleophile, including by [¹⁸F]F⁻; and
an integer number n of atoms or functional groups Z covalently bound to Q,
wherein n≥0, and each Z is chemically inert and independently chosen such that it stabilizes Q or otherwise protects the functionality from decomposition; and
the sum of m, n, and 1 is a value that corresponds to a coordination number specific to Q, wherein the coordination number for Si can be one of: 4, 5, or 6; and wherein the compound comprises:
a handle moiety operatively coupled to the aromatic heterocyclic core, wherein:
the handle moiety is adapted to couple the compound to a ligand,
the handle moiety comprises a functional group selected from the group consisting of: an activated ester, a N-hydroxysuccinimide ester, a maleimide, an aldehyde, a thiol, a nitrile, a disulfide, an alcohol, an isocyanate, a isothiocyanate, an aryl halide, a benzoyl halide, an amine, an azide, an alkyne, a tetrazine, a strained alkyne or a carboxylic acid; and
a linker operatively coupled to the aromatic heterocyclic core, wherein:
the linker is adapted to link the aromatic heterocyclic core to the ligand,
the linker moiety comprises a functional group selected from an unsubstituted alkyl; an unsubstituted polyethylene glycol, a charged or neutral polyamine; a mixed amino-oxo chain; a polyaromatic polyheteroaromatic group, a charged or neutral polyheteroaromatic group; a bi- or poly-substituted triazole; an imidazole containing group; a peptide, an or amino acid containing moiety or combinations thereof.

2. The composition of claim 1, wherein the compound is of the general formula: 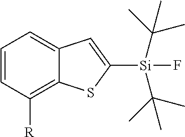
wherein R comprises the handle moiety.

3. The composition of claim 2, further comprising:
a chelator; and/or
a polar auxiliary moiety operatively coupled to the compound; wherein the chelator or the polar auxiliary moiety functions to modulate hydrophilicity such that the polar auxiliary moiety exhibits a negative charge at physiological pH.

4. The composition of claim 3, wherein the chelator comprises an optionally metalated hydrophilic metal chelator.

5. The composition of claim 3, wherein the handle moiety comprises a carboxylic acid, the linker comprises a polyethylene glycol and the chelator comprises DOTA (1,4,7,10-tetraazacyclododecane-1,4,7,10-tetraacetic acid) or NOTA (1,4,7-Triazacyclononane-1,4,7-triacetic acid).

6. The composition of claim 1, wherein the compound has the general formula: 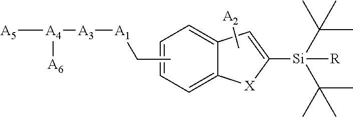
wherein:
A1 comprises the handle moiety;
A3 comprises an unsubstituted polyethylene glycol or a bisubstituted triazole;
A4 comprises the ligand;
A5 comprises a chelator;
A6 comprises a polar auxiliary moiety; and
R comprises a fluorine atom.

7. The composition of claim 1, wherein the compound is coupled to a ligand comprising a peptide, a protein, an enzyme or a small molecule having a molecular weight less than 900 Daltons.

8. A method of making a heteroaromatic silicon-fluoride compound comprising a [¹⁸F] atom, the method comprising:
disposing a [¹⁸F]fluoride donor compound within a cartridge comprising a quaternary methyl ammonium so that a [¹⁸F] tetraethyl ammonium fluoride compound is formed;
eluting the [¹⁸F] tetraethyl ammonium fluoride compound from the cartridge with a solution comprising Tetraethylammonium bicarbonate at a concentration less than 50 umol;
drying the eluted [¹⁸F] tetraethyl ammonium fluoride compound;
combining a heteroaromatic silicon-fluoride compound precursor compound comprising an [¹⁹F] atom with the dry [¹⁸F] tetraethyl ammonium fluoride compound in an organic solvent (acetonitrile) so that the heteroaromatic silicon-fluoride acceptor compound and the dry [¹⁸F]tetraethyl ammonium fluoride compound exchange F isotopes; and
quenching the isotope-exchange reaction with water;
such that the heteroaromatic silicon-fluoride compound comprising the ¹⁸F atom is made.

9. The method of claim 8, wherein the [¹⁸F] tetraethyl ammonium fluoride compound is eluted from the cartridge with a solution comprising Tetraethylammonium bicarbonate at a concentration between 5 umol and 15 umol.

10. The method of claim 8, wherein the method is performed at room temperature.

11. The method of claim 8, wherein the method obtains a radiochemical conversion of at least 80%.

12. The method of claim 8, wherein the heteroaromatic silicon-fluoride acceptor compound is combined with the dry [¹⁸F] tetraethyl ammonium fluoride compound in the organic solvent for not more than 10 minutes.

13. The method of claim 8, wherein the heteroaromatic silicon-fluoride precursor compound comprises a compound of claim 1.

14. The method of claim 13, wherein the ligand is coupled to the compound at a moiety on position 7 of the aromatic heterocyclic core.

15. A method for imaging a biological target by positron emission tomography, the method comprising:
introducing into the target an imaging agent comprising: a composition of claim 7, wherein F is ¹⁸F; and
imaging the target by a positron emission tomography process such that the biological target is imaged by positron emission tomography.

16. The method of claim 15, wherein the imaging agent is obtained by site selective chemical conjugation of the ligand to the compound of claim 1.

17. The method of claim 15, wherein the chemical conjugation of the ligand to the compound occurs via carboxylic acid moiety on position 7 of the aromatic heterocyclic core.

18. The method of claim 15, wherein the biological target comprises a human organ, human tissue or human cancer cells.

19. A kit for ¹⁸F-labeling of a compound of claim 1, the kit comprising a compound of claim 1 wherein F is ¹⁹F, an ¹⁸F isotopic exchange reagent, and an instruction manual for the use thereof.

20. The kit of claim 19, wherein the kit comprises a container comprising a nonpolar solution comprising Tetraethylammonium bicarbonate at a concentration between 1 umol and 20 umol.
